(19) United States
(12) Patent Application Publication
(10) Pub. No.: US 2014/0023692 A1
(43) Pub. Date: Jan. 23, 2014

(54) DRUG DELIVERY DEVICE

(71) Applicants: Lisa Claire Du Toit, Johannesburg (ZA); Viness Pillay, Johannesburg (ZA); Yahya Essop Choonara, Johannesburg (ZA); Thirumala Govender, Mount Edgecombe (ZA); Trevor Robin Carmichael, Johannesburg (ZA)

(72) Inventors: Lisa Claire Du Toit, Johannesburg (ZA); Viness Pillay, Johannesburg (ZA); Yahya Essop Choonara, Johannesburg (ZA); Thirumala Govender, Mount Edgecombe (ZA); Trevor Robin Carmichael, Johannesburg (ZA)

(21) Appl. No.: 13/989,401

(22) PCT Filed: Nov. 28, 2011
(86) PCT No.: PCT/IB2011/055328

§ 371 Date: Sep. 5, 2013

(30) Foreign Application Priority Data
Nov. 26, 2010 (ZA) 2010/03748

(51) Int. Cl. A61K 9/00; A61K 31/496; A61K 45/06; A61K 31/405

(52) U.S. Cl. CPC A61K 9/0051; A61K 31/405; A61K 31/496; A61K 45/06
USPC 424/428; 514/420; 514/253.08; 264/113

(57) ABSTRACT

The invention provides an inflammation-responsive implantable device for the in situ delivery of one or more pharmaceutically active agents to a human or animal. The device comprises two differential release bioresponsive polymeric matrices (BPMs): an outer polymetric matrix and an inner polymeric matrix, both of which contain at least one pharmaceutically active agent or drug, typically an antibiotic and an anti-inflammatory agent, respectively. The therapeutically effective agent may be embedded in nanoparticles or nanobubbles. In response to inflammation, the pharmaceutically active agents are released, but at different rates: the rate of drug release from the inner polymeric matrix is lower than the rate of drug release from the outer polymeric matrix. Suitable polymers for forming the outer and inner polymeric matrices are hyaluronic acid and chitosan, respectively. A method of making the device and a method of treatment are also described.

### FIELD OF THE INVENTION

This invention relates to an implantable device for the in situ delivery of one or more pharmaceutically active agents for acute and chronic management of inflammation and/or infection.

### BACKGROUND TO THE INVENTION

On the basis of data from surveys in 55 countries, the World Health Organization has estimated that there are approximately 161 million people in the world with visual impairments and 37 million blind people. The pertinence of treating intraocular pathologies before blindness manifests is apparent.

In their investigations, Herrero-Vanrell and Refojo (2001) and Del Amo and Urtti (2008) have pointed to inflammatory posterior segment ocular (vitreoretinal) disorders as the foremost perpetrators of visual impairment and ultimately blindness. Ensuring delivery of the indicated bioactive to the posterior segment of the eye is fundamental for the effectual treatment of internal eye structure disorders. However, drug delivery to the posterior segment is particularly challenging due to the anatomical and vascular barriers to both local and systemic access.

As emphasised by Yasukawa and co-workers (2005), progress in the field of ocular drug delivery is delayed when problems of drug availability to the posterior segment are encountered. Furthermore, Haesslein et al. (2006) reiterated that indirect bioactive pathways (topical, systemic or periocular) to the vitreous suffer from the disadvantage of poor penetration of the biophysiological blood-ocular barriers, necessitating direct intravitreal drug delivery for successful management of posterior segment disorders. Intravitreal injection of drug, and sustained drug delivery systems fabricated from polymers (biodegradable or non-biodegradable) for delivery via injection or implantation. target the posterior segment.

Because there may be significant damage to retinal and uveal tissues, Rao (1990) noted that visual prognosis is most critical where severe intraocular inflammation is a presenting feature; the process is initiated by T- and B-lymphocytes, but augmented and maintained by polymorphonuclear leukocytes (PMNs) and macrophages. Chemical mediators, such as arachidonic acid metabolites, proteolytic enzymes and oxygen metabolites are responsible for the tissue damage evident in ocular inflammatory conditions such as uveitis (infectious or non-infectious). The emerging focus on reactive oxygen metabolites (oxygen free radicals) released by PMNs and macrophages during the initial phase of inflammation was highlighted by Rao (1990). Champagne (2001) specified the topical and systemic use of corticosteroids and nonsteroidal anti-inflammatory drugs (NSAIDs) for the management of adnexal, corneal and intraocular inflammation. Corticosteroid suppression of inflammation and cicatrisation is reiterated by Holekamp et al. (2005) attained in part by their inhibition of inflammatory cytokines. Intravitreal corticosteroids (e.g. dexamethasone, fluocinolone acetonide, triamcinolone) are purported to result in improvements in patients with many chronic, inflammatory and proliferative intraocular diseases (Haesslein et al., 2006; Reichle et al., 2005) such as macular oedema secondary to diabetes (Jonas and Sofker, 2001), pseudophakia (Jonas et al., 2003), central retinal vein occlusion (Park et al., 2003) and uveitis (Young et al., 2001); as well as in the prevention of proliferative vitreoretinopathy (Jonas et al., 2000). NSAIDs (e.g. flurbiprofen, keratolac, acetylsalicylic acid) are being used with increasing frequency, with exploration of further applications (Champagne, 2001).

Posterior segment pathologies further encompass intraocular infections, e.g. bacterial endophthalmitis, which can occur post-operatively, post-traumatically or via bacterial metastasis from an endogenous site. The clinical presentation of endophthalmitis varies from mild inflammation to complete loss of vision or loss of the eye (Callegan at al., 2007). Callegan and co-workers (2007) referred to experimental evidence, demonstrating the necessity to initiate treatment with intravitreal antibiotics in a timely manner. Vancomycin, aminoglycosides, cephalosporins or the promising fourth generation fluoroquinolones, are often used empirically, with corticosteroids as an adjunct to limit the bystander damage caused by intraocular inflammation.

Despite these advances, the pharmacological management of these severe ocular pathologies is still a major hurdle. There is widespread procedural occurrence (Reichle et al., 2005) of elevated intraocular pressure and cataract progression (Roth et al., 2003). Other risks, particularly associated with intravitreal injection of corticosteroids, include endophthalmitis (Moshfeghi et al., 2003), retinal detachment (Jonas et al., 2003), hemicentral vein occlusion (Gillies et al., 2004), preretinal haemorrhage (Jonas et al., 2001), pseudohypopyon (Jonas et al., 2001) and vitreous haemorrhage (Moshfeghi et al., 2003). Furthermore, the combination of antibiotic and corticosteroid in the therapeutic management of endophthalmitis is still controversial due to corticosteroid-related effects (Callegan et al., 2005). Research has been implicit in conveying that controlled polymeric drug delivery systems are essential for realising a superlative pharmaceutical intervention, where effective bioactives are available for intraocular disease treatment. Such systems attain ‘controlled’ levels of drug, for bioavailability optimisation and side effect minimization (Ligório Fialho et al., 2008). However, available intraocular implants for high-dose sustained corticosteroid delivery suffered from a notably high complication rate during clinical trials conducted by Holekamp et al. (2005).

Retisert™ (fluocinolone 0.59 mg intravitreal implant. Bausch and Lomb, Inc.) is the first FDA approved intravitreal implant for the treatment of chronic posterior non-infectious uveitis. It is a sterile implant that releases fluocinolone initially at a rate of 0.6 micrograms per day to the posterior segment of the eye, decreasing over the month to 0.3-0.4 micrograms per day over approximately 30 months. Because there is continuous release of anti-inflammatory drug, irrespective of the presence or absence of inflammation, there is an enhanced propensity for the occurrence of side effects, such as cataract development, intraocular pressure elevation, procedural complications and eye pain. This would be minimized from the proposed system which provides enhanced drug release when exposed to an inflammatory stimulus compared to when the implant is subjected to normal intraocular conditions.

There is therefore a need for a means of delivering drugs which overcomes at least some of the problems highlighted above.

### SUMMARY OF THE INVENTION

According to a first embodiment of the invention, there is provided an implantable device for the in situ delivery of one or more pharmaceutically active agents to a human or animal, the device comprising:
an outer polymeric matrix formed from at least one inflammation-sensitive polymer and comprising at least one pharmaceutically active agent, wherein the polymer is cross-linked so as to retain the pharmaceutically active agent within the polymeric matrix under normal physiological conditions but undergoes a conformational change when inflammation is present so as to release the pharmaceutical composition; and
an inner polymeric matrix formed from at least one inflammation-sensitive polymer and comprising at least one pharmaceutically active agent, wherein the polymer is cross-linked so as to retain the pharmaceutically active agent within the polymeric matrix under normal physiological conditions but undergoes a conformational change when inflammation is present so as to release the pharmaceutical composition;
wherein the inner and outer polymeric matrices are formed so that, when inflammation is present, the pharmaceutically active agent within the inner polymeric matrix is released at a slower rate than the pharmaceutically active agent within the outer polymeric matrix.

The outer polymeric matrix may provide fast to intermediate release of the pharmaceutically active agent for the therapeutic management of infection and/or preliminary inflammation, and the inner polymeric matrix may provide slower release of the pharmaceutically active agent than the outer polymeric matrix for chronic responsive management of inflammation.

The inner polymeric matrix may be chemically modified by cross-linking to provide a slower release rate of the pharmaceutically active agent than the outer polymeric matrix.

The device may be biodegradable.

The polymer of the inner polymeric matrix may be a cationic low-molecular weight carbohydrate polymer, such as chitosan.

The polymer of the outer polymeric matrix may be an anionic polymer, such as hyaluronic acid, or a mixture of anionic polymers.

The polymers of the inner and outer polymeric matrices may be eroded by free radicals released from activated leukocytes during acute and chronic intraocular inflammatory reactions.

The polymeric matrices may be cross-linked with gluteraldehyde and may be further cross-linked (double-cross-linked) employing carbodiimide coupling chemistry.

The outer polymeric matrix may further comprise alginate, polygalacturonate, methylcellulose (polyacetals), poly (ethylene) oxide and/or poly (acrylic acid). The ratio of alginate:hyaluronic acid may be about 16:1. The ratio of alginate:poly (acrylic acid) in the outer polymeric matrix may be about 4:1.

The ratio of chitosan to the gluteraldehyde cross-linking agent in the inner polymeric matrix may be about 7:1.

The pharmaceutically active agent of the outer polymeric matrix may be an antibiotic, such as ciprofloxacin or other fluoroquinolones (e.g. moxifloxacin, gatifloxacin, levofloxacin), or other suitable antibiotics or antifungal agents (e.g. vancomycin, amikacin, gentamicin, tobramycin, ceftazidime, Amphotericin B) or may be an anti-inflammatory agent.

The pharmaceutically active agent of the inner polymeric matrix may be an anti-inflammatory agent (steroidal or non-steroidal).

The anti-inflammatory agent may be the non-steroidal agent, indomethacin.

The pharmaceutically active agent in the inner polymeric matrix may be within or on nanoparticles. The nanoparticles may be formed from poly(c-caprolactone), chitosan and phospholipids, and may be in the form of nanobubbles. The nanoparticles may possess the inherent potential to permeate ocular barriers of interest, such as the blood-retinal barrier (BRB).

The device may have at least one aperture for suturing the implant to a site in the body.

The device may be an intraocular device for implantation or insertion into the eye, preferably into the posterior segment of the eye (at the pars plana) or sub-Tenon, or intrasclerally or on the sclera. Alternatively, the device may be for use in preventing or treating inflammatory or infectious conditions throughout the body, such as HIV/AIDS, influenza, arthritis, lupus, fibromyalgia, juvenile rheumatoid arthritis, osteomyelitis or septic (infectious) arthritis. It may also be applied in the management of chronic pain associated with cancer. It may therefore be implanted in regions other than the eye.

In a preferred example:
the polymer with which the outer polymeric matrix is formed is hyaluronic acid;
the pharmaceutically active agent in the outer polymeric matrix is an antibiotic;
the polymer with which the inner polymeric matrix is formed is chitosan;
the pharmaceutically agent in the inner polymeric matrix is an anti-inflammatory agent; and
the anti-inflammatory agent is entrapped in or on nano-particles.

According to a second embodiment of the invention, there is provided a method of manufacturing a device as described above, the method comprising the steps of
forming nanoparticles from poly(c-caprolactone), chitosan, phospholipids and a pharmaceutically active agent;
forming an inner polymeric matrix from the nanoparticles and a polymer which erodes when exposed to inflammation;
forming an outer polymeric matrix from a pharmaceutically active agent and a polymer which erodes when exposed to inflammation, wherein the outer polymeric matrix is designed to erode at a faster rate than the inner polymeric matrix when exposed to inflammation, and so to release the pharmaceutically active agent from the outer polymeric matrix at a faster rate than the inner polymeric matrix;
placing the inner polymeric matrix in an inner portion of a mould;
placing the outer polymeric matrix in an outer portion of the mould;
drying the matrices to form a solid device which is suitable for implantation; and
optionally creating apertures in the device to enable it to be sutured to a site in the body.

According to a third embodiment of the invention, there is provided a method of treating infection and/or inflammation in a human or animal, the method comprising inserting or implanting a device as described above into the human or animal at a site to be treated, wherein:
an outer polymeric matrix of the device releases, in the presence of inflammation, a therapeutically effective amount of an antibiotic to treat the infection and preliminary inflammation; and
an inner polymeric matrix of the device releases a therapeutically effective amount of an anti-inflammatory agent at a slower rate than the outer polymeric matrix to treat a chronic inflammatory condition.

### BRIEF DESCRIPTION OF THE FIGURES

FIG. 1: shows the proposed configuration of an implant device according to the invention possessing a ‘fried egg’ appearance with inclusion of optional apertures, created employing a laser or tabletting press; (a) front view, (b) lateral view.

FIG. 2: shows a constructed multilayer perceptron network. An artificial neural network is an interconnected group of nodes, akin to the vast network of neurons in the human brain.

FIG. 3: shows photographic images depicting (a) simultaneous origination of bioresponsive polymetric matrices (BPMs) of the device according to the invention, and (b) the final device and resultant diameter.

FIG. 4: shows exemplary graphical depictions of the correlation between the WAC and MDT of the device under normal and inflammatory conditions representing: (a) high correlation under inflammatory conditions (Formulation 10), (b) high correlation under normal conditions (Formulation 20), and (c) high correlation under normal and inflammatory conditions (Formulation 24).

FIG. 5: shows drug release profiles for formulations 1-6 (a-f) (SD<±0.03042 for indomethacin and SD<±0.05607 for ciprofloxacin in all cases).

FIG. 6: shows drug release profiles of formulations 7-12 (SD<±0.03042 for indomethacin and SD<±0.05607 for ciprofloxacin in all cases).

FIG. 7: shows drug release profiles for formulations 13, 14, 16-18, 20 (SD<*0.03042 for indomethacin and SD<±0.05607 for ciprofloxacin in all cases).

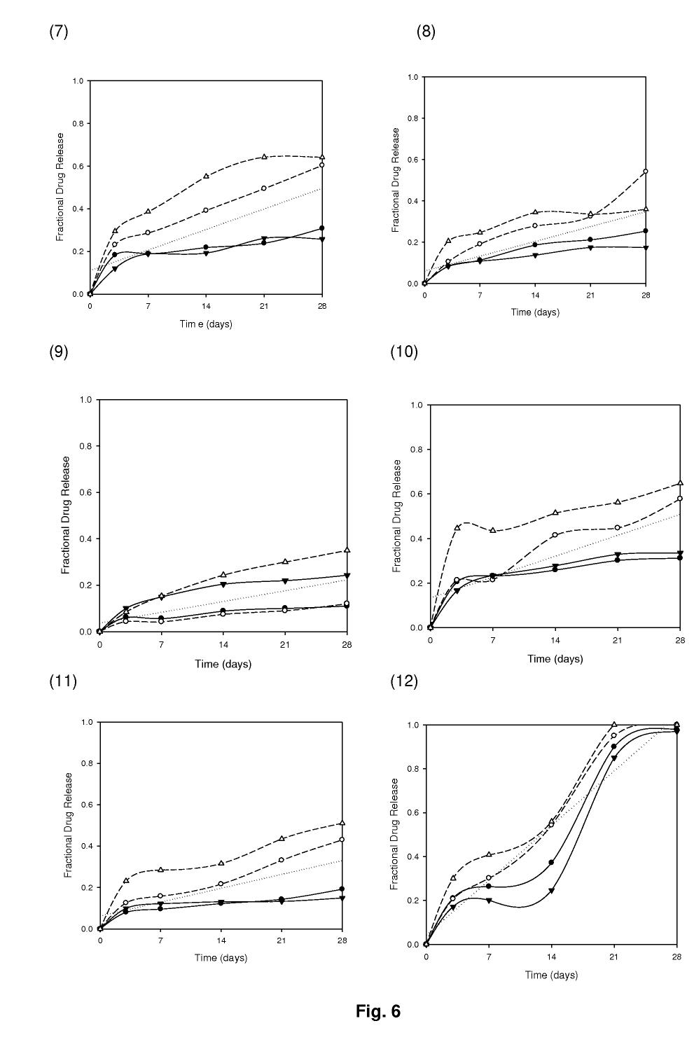

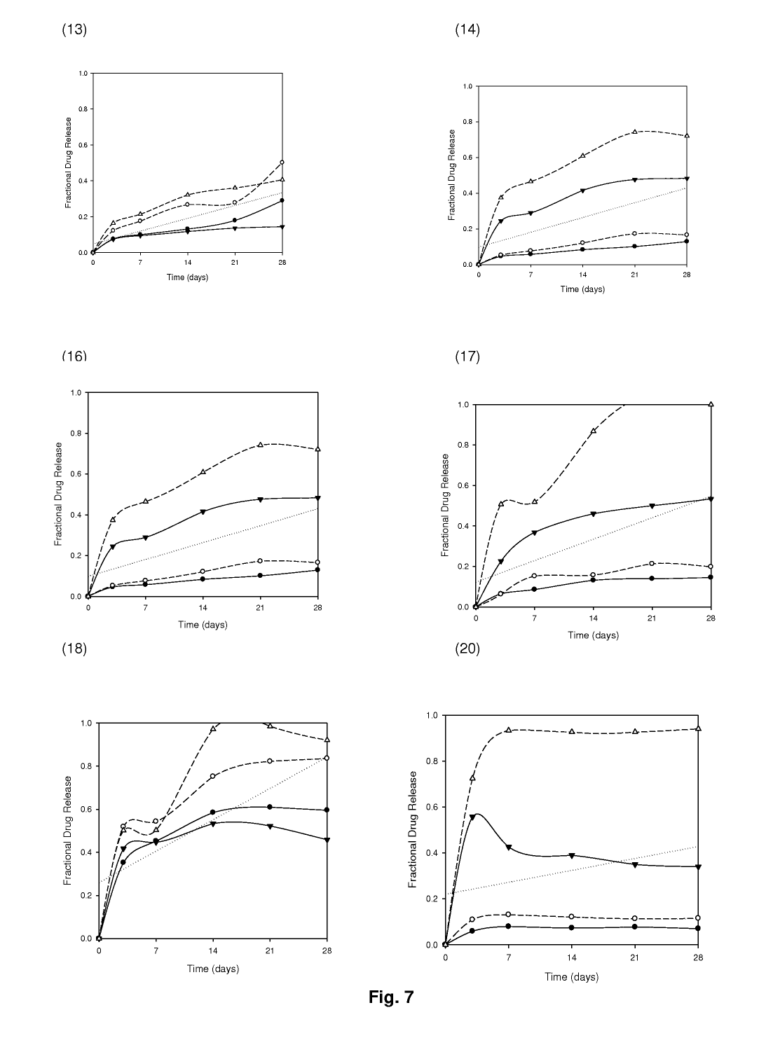

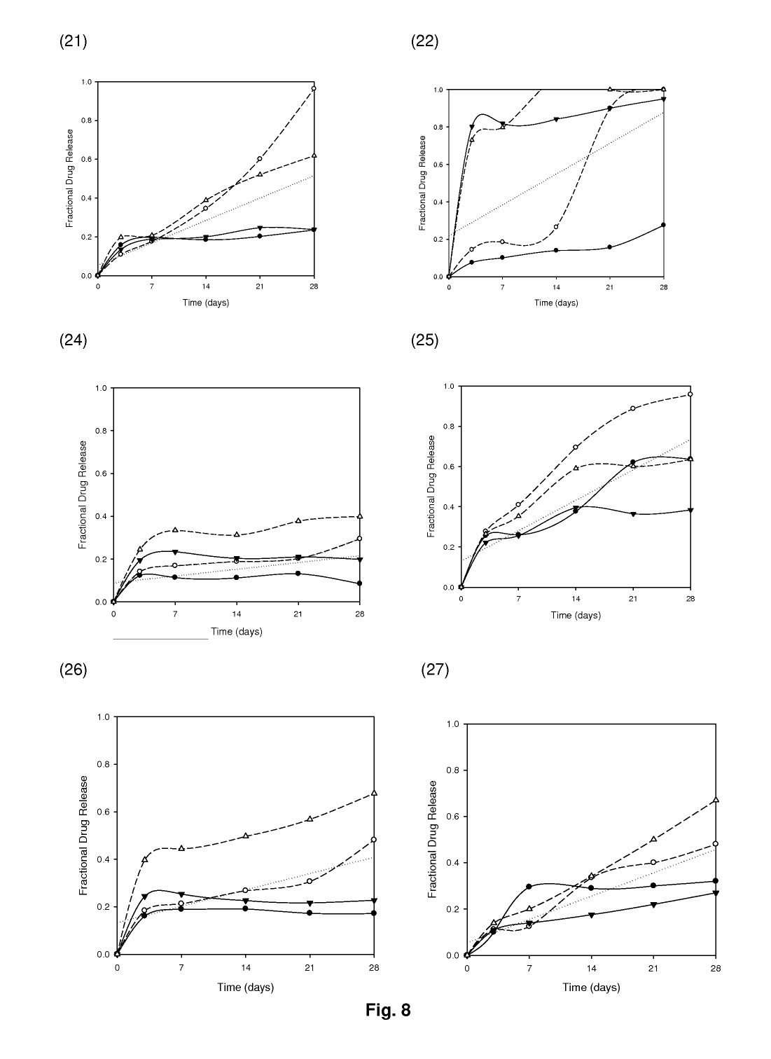

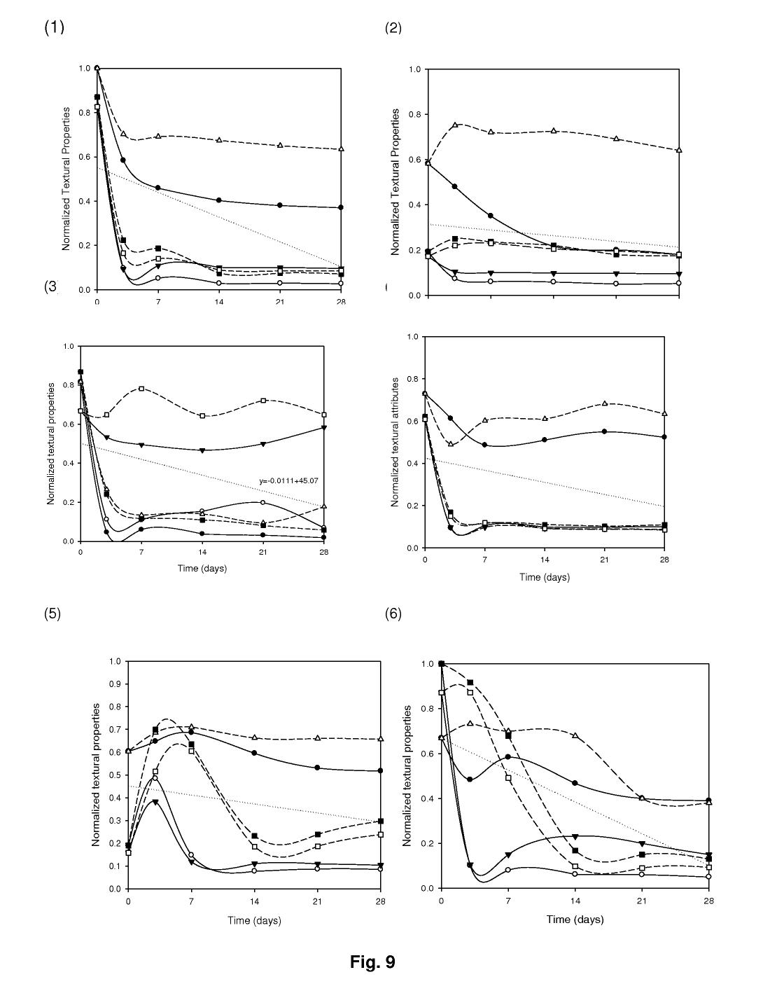

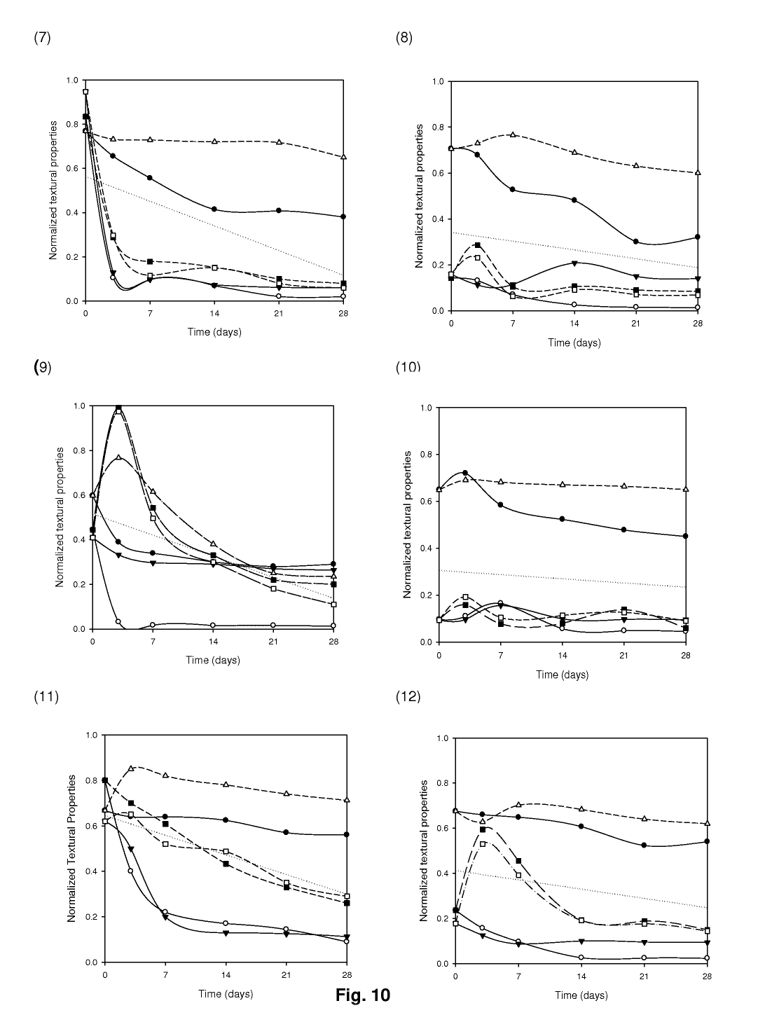

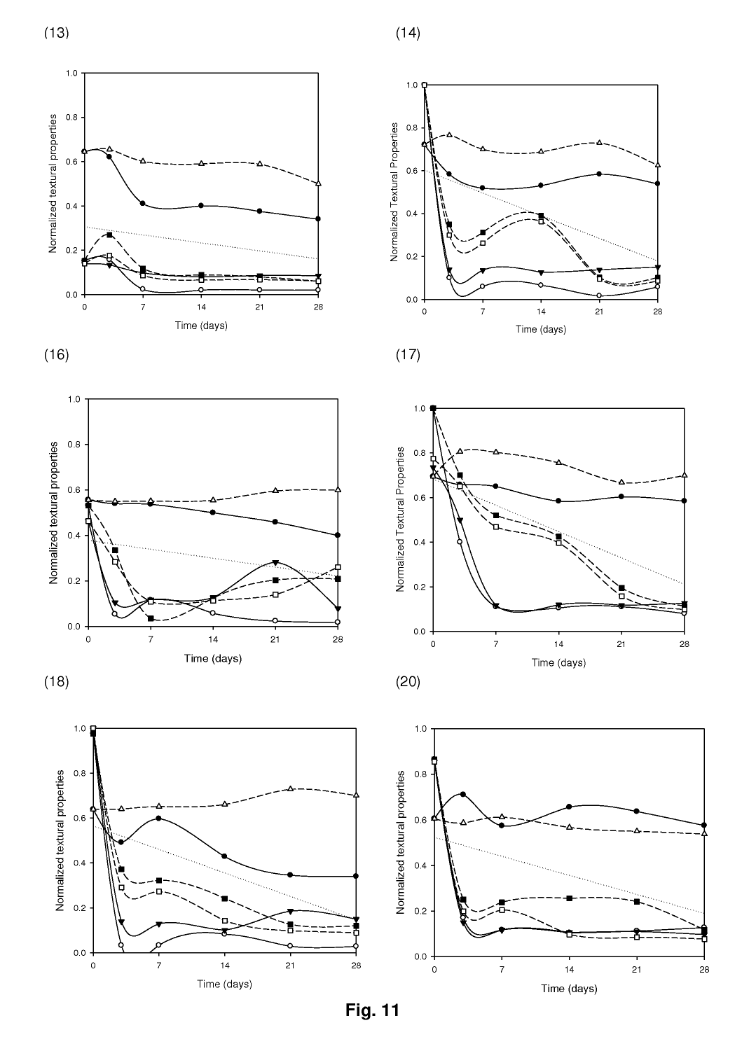

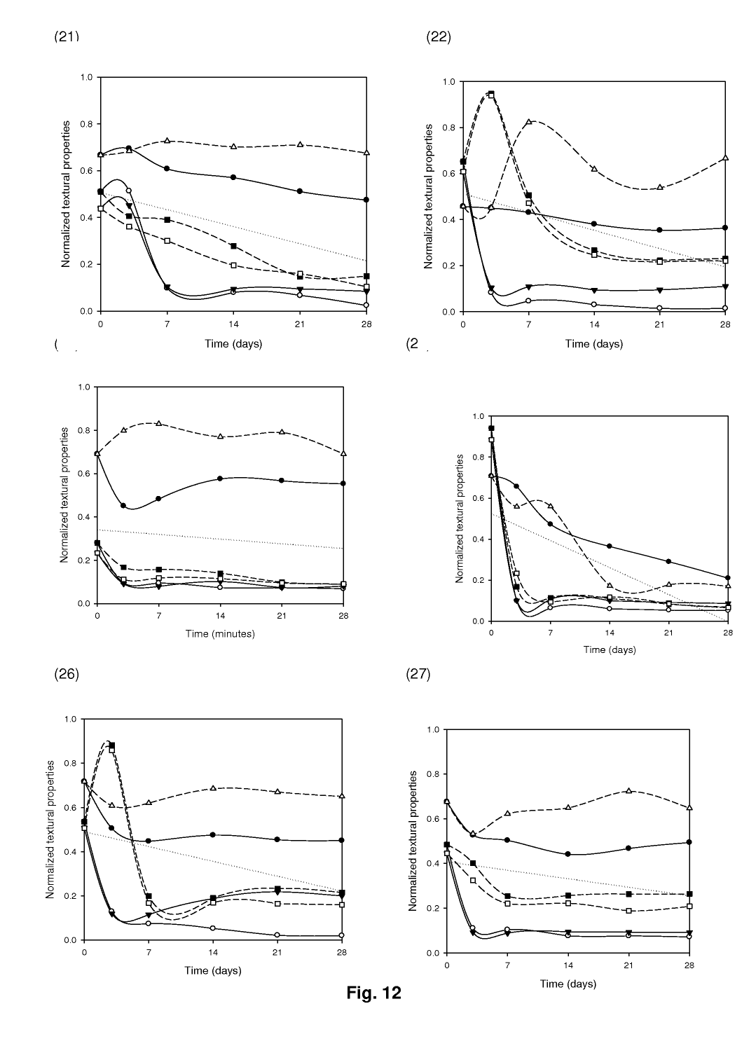

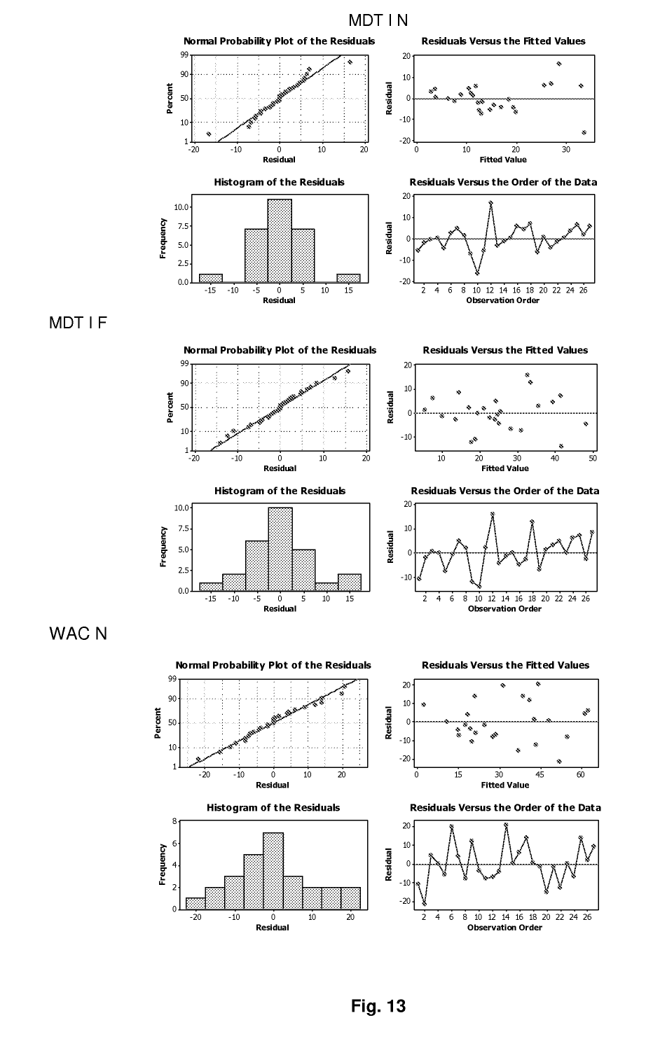

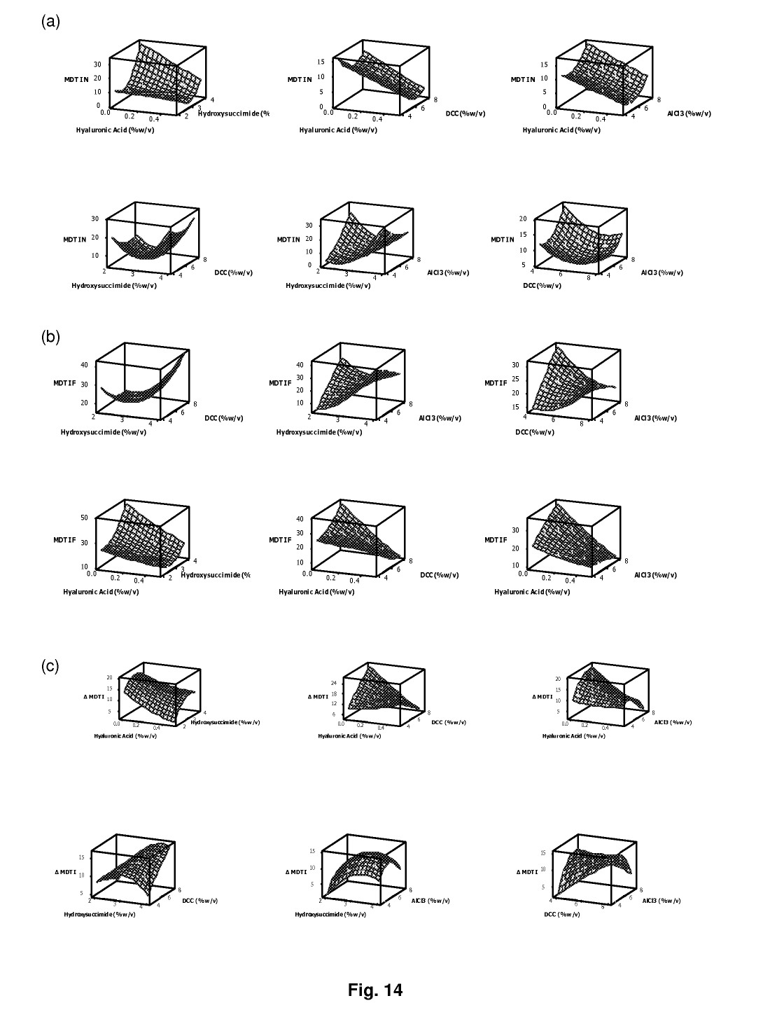

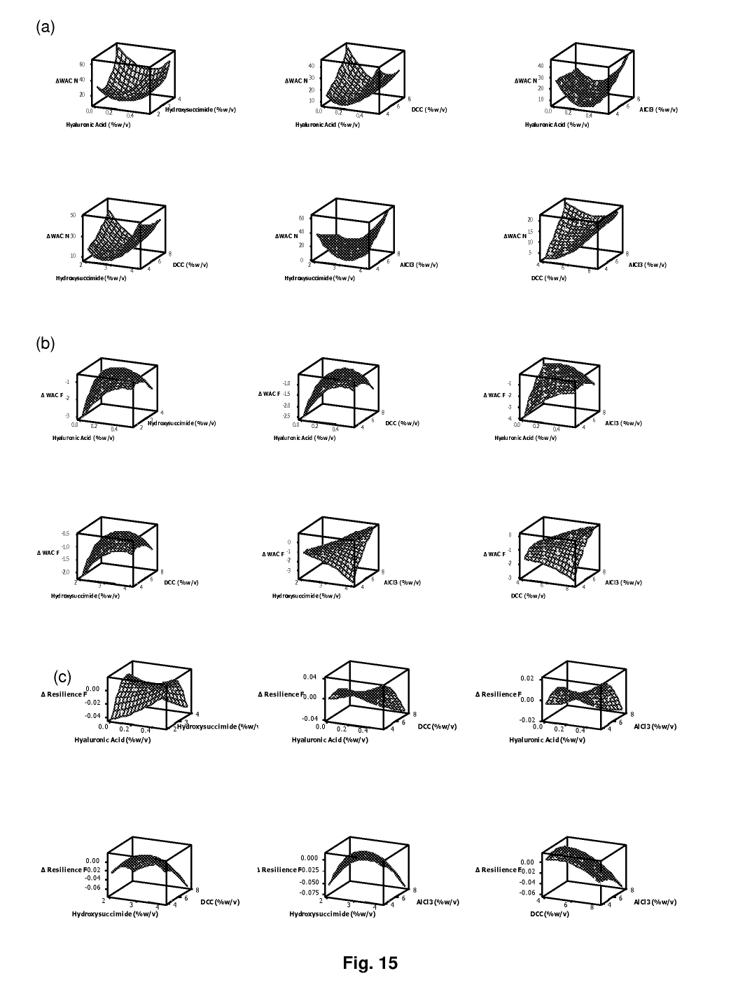

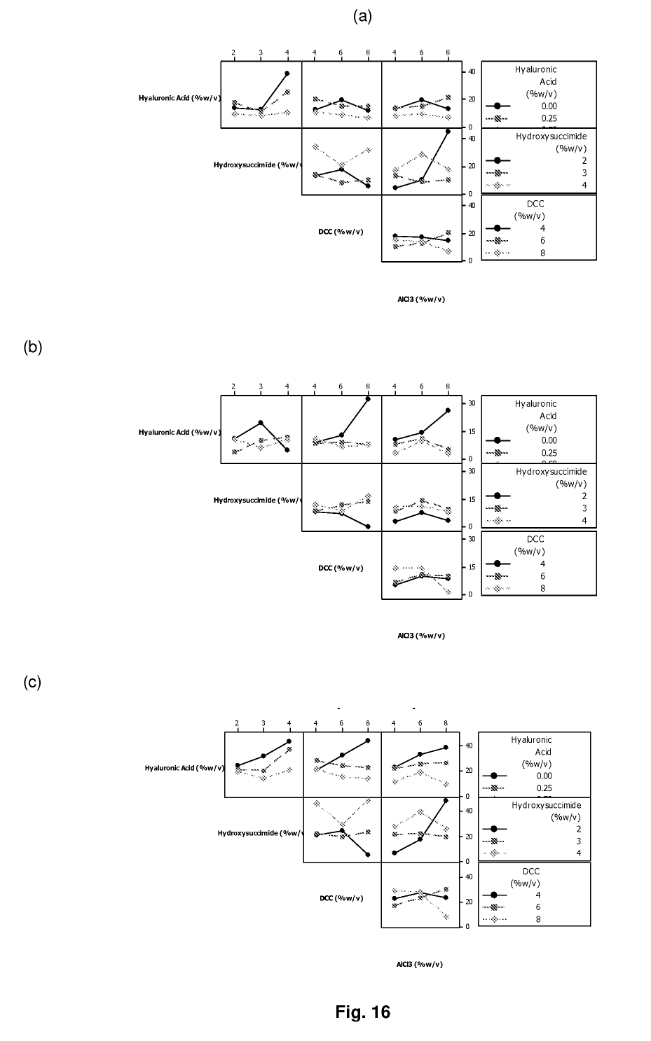

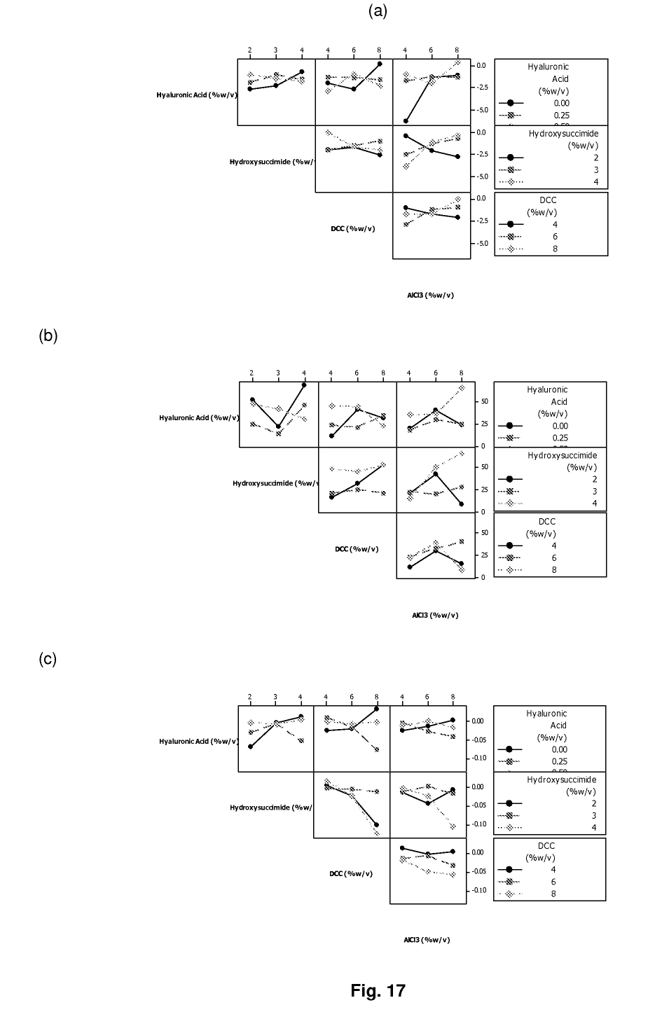

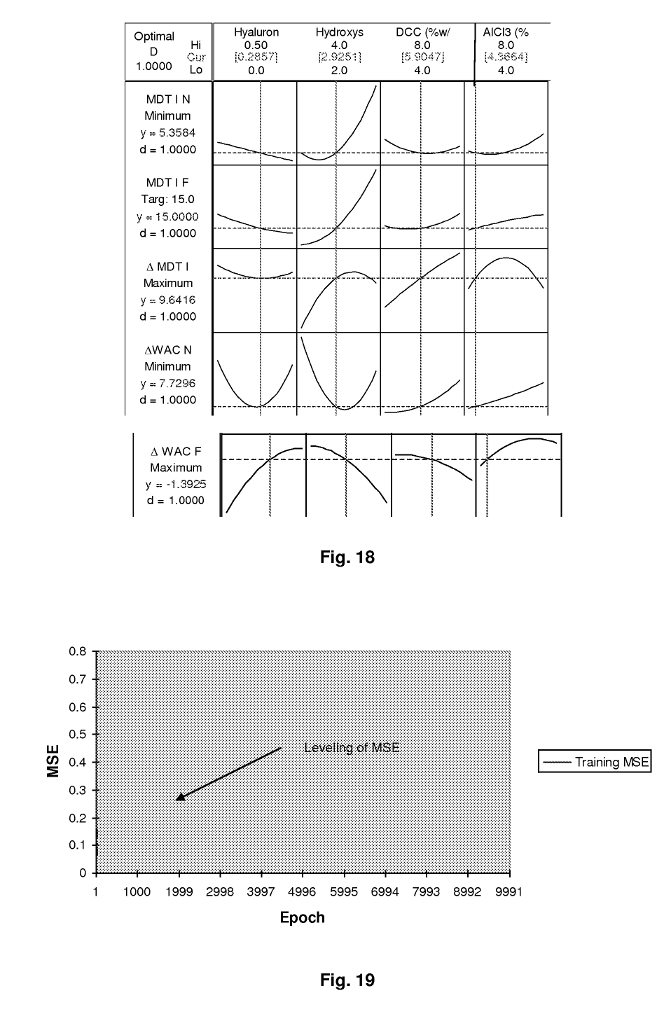

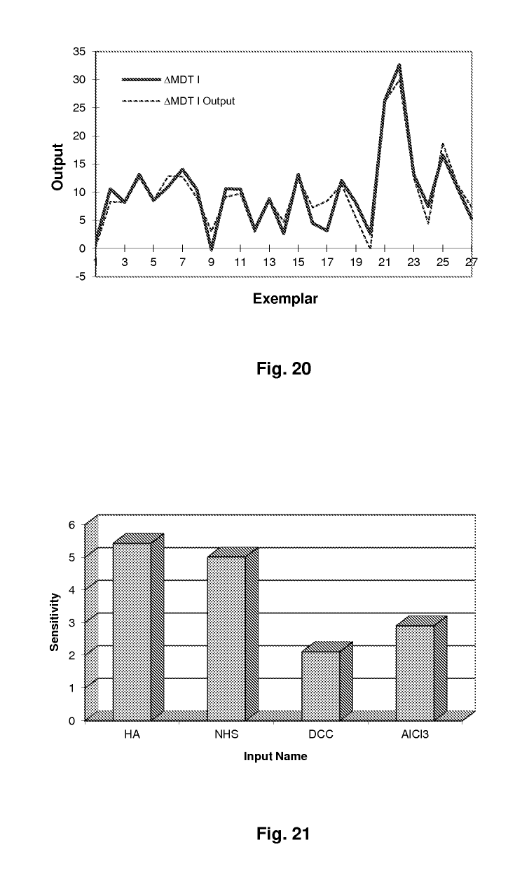

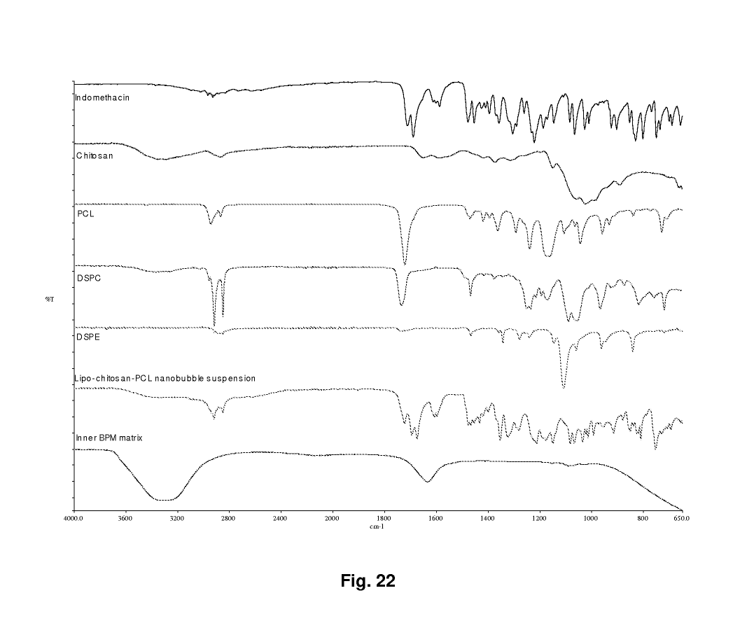

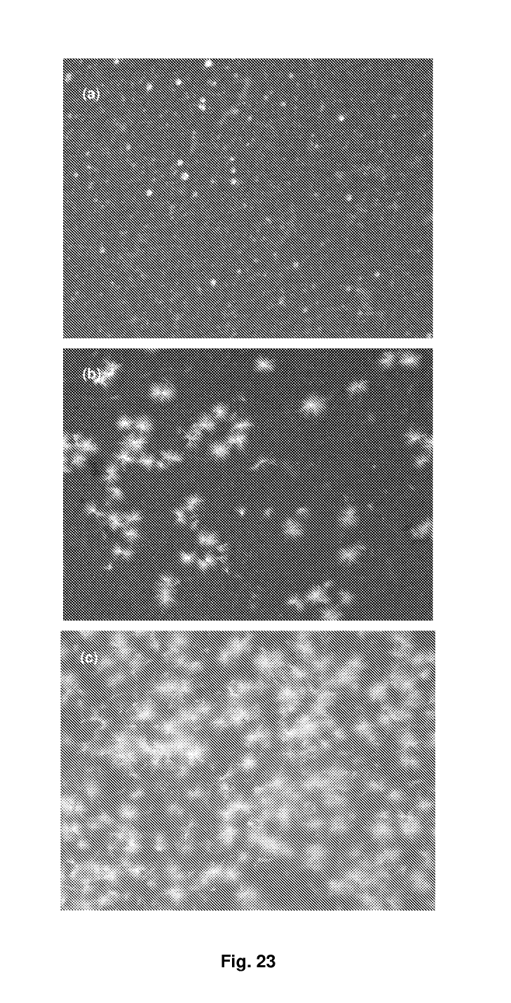

FIG. 6: shows drug release profiles for formulations 21, 22, 24-27 (SD<±0.03042 for indomethacin and SD<±0.05607 for ciprofloxacin in all cases).

FIG. 9: shows normalized transitional textural profiles for formulations 1-6 (S.D.<±0.08112 in all cases).

FIG. 10: shows normalized transitional textural profiles for formulations 7-12 (S.D.<±0.08112 in all cases).

FIG. 11: shows normalized transitional textural profiles for formulations 13, 14, 16-18, 20 (S.D.<±0.08112 in all cases).

FIG. 12: shows normalized transitional textural profiles for formulations 21, 22, 24-27 (S.D.<±0.08112 in all cases).

FIG. 13: shows exemplary residual plots for MDT I N, MDT I F, and WAC N.

FIG. 14: shows response surface plots for the significant responses (a) MDT I N, (b) MDT I F, (c) Δ MDT I.

FIG. 15: shows response surface plots for the significant responses (a) Δ WAC N (b) Δ WAC F (c) Δ Resilience F.

FIG. 16: shows interaction plots for (a) MDT I N, (b) MDT I F and (c) change in MDT I.

FIG. 17: shows interaction plots (data means) for (a) change in WAC (N), (b) change in WAC (F), and (c) change in resilience (F).

FIG. 18: shows optimization plots delineating factor settings and desirability values for an optimal formulation.

FIG. 19: shows a graphical depiction of the training performed on Neurosolutions™.

FIG. 20: shoes a graphical depiction of the correlation between the desired and the actual network output for Δ MDT I of each formulation.

FIG. 21: shows a typical bar chart graph depicting the sensitivity coefficients (sensitivity about the mean) of each variable implicated in the manufacture of the device against the ΔMDT I following the primary training.

FIG. 22: shows FTIR spectra of the drug, polymers, lipids, and the resultant nanobubble.

FIG. 23: shows progressive deposition of the polysaccharide coat on the lipo-chitosan-PCL nanobubbles at 32× magnification: (a) the uncoated chitosan-PCL nanobubbles, with time the coating emanated in the elucidation of fuzzy microstructures as viewed at (b) 12 hours and (c) 24 hours.

FIG. 24: shows SEMs depicting that artefacts of the nanobubbles (pores previously occupied by the nanobubbles) can be visualised (4450× magnification).

FIG. 25: shows computational data depicting: (a) polymer strands ordering under external influence: A) polymer strands in solution with recognizable molecular sites; B) initial ordering around axis (taken as start up point only) with surfactant's addition to the medium; C, D, E & F) further ordering and a complete three-dimensional, 360° ordering orientation of the polymer strands. (b) Orientation progression for 5a. (c) Polymer strands ordering under external influence: A) polymer strands in solution with recognizable molecular sites; B) initial ordering around axis (taken as start up point only) with surfactant's addition to the medium; C, D, E & F) further ordering and a complete three-dimensional, 360° ordering orientation of the polymer strands.

FIG. 26: shows FTIR spectra of the native polymers, pre-crosslinked gel implicated in formation of the outer BPM, and the crosslinked BPM.

### DETAILED DESCRIPTION OF THE INVENTION

The invention provides an implantable device for the in situ delivery of one or more pharmaceutically active agents to a human or animal. The device comprises two differential release bioresponsive polymeric matrices (BPMs): an outer polymetric matrix and an inner polymeric matrix, both of which contain at least one pharmaceutically active agent or drug, typically an antibiotic and an anti-inflammatory agent, respectively. In response to inflammation, the pharmaceutically active agents are released, but at different rates: the rate of drug release from the inner polymeric matrix is lower than the rate of drug release from the outer polymeric matrix. A number of inflammatory diseases are chronic and hence require prolonged drug therapy. The outer polymeric matrix is designed for intermediate drug release for the therapeutic management of the infection and/or the preliminary inflammatory reaction, while the inner polymeric matrix is designed for chronic responsive management of the ensuing inflammatory condition.

The invention will be described below with reference to treating infection and inflammation in the eye. However, it will be apparent to a person skilled in the art that the device can also be implanted or inserted into other regions of the body. For example, the device could be used to treat inflammatory and/or infectious afflictions ranging from HIV/AIDS and influenza to arthritis, lupus and fibromyalgia. The device could also be of considerable value for a number of inflammatory and infectious disorders that affect the body's musculoskeletal system, including juvenile rheumatoid arthritis, osteomyelitis and septic (infectious) arthritis.

The sclera is the outermost coat of the eye, covering the posterior portion of the globe. The external surface of the scleral shell is covered by an episcleral vascular coat, by Tenon's capsule, and by the conjunctiva. The tendons of the six extraocular muscles insert into the superficial scleral collagen fibres. Numerous blood vessels pierce the sclera through emissaria to supply as well as drain the choroid, ciliary body, optic nerve, and iris. Inside the scleral shell, the vascular choroid nourishes the outer retina by a capillary system in the choriocapillaris. Bruch's membrane and the retinal pigment epithelium (RPE) are situated between the outer retina and the choriocapillaris; their tight junctions provide an outer barrier between the retina and the choroid. The multifunctional RPE is implicated in vitamin A metabolism, phagocytosis of the rod outer segments, and multiple transport processes.

The neurosensory retina, the most extensively investigated structure of the eye, is a thin, transparent, highly organized structure of neurons, glial cells, and blood vessels. Notably, the unique organisation and biochemistry of the photoreceptors is a superb model system for investigating signal transduction mechanisms. The wealth of information about rhodopsin has made it an excellent model for the G protein-coupled signal transduction.

The optic nerve is a myelinated nerve conducting the retinal output to the central nervous system. It is composed of: 1) an intraocular portion, which is visible as the 1.5 mm optic disk in the retina; 2) an intraorbital portion; 3) an intracanalicular portion; and 4) an intracranial portion. The nerve is ensheathed in meninges continuous with the brain (Henderer and Rapuano).

The following facts are thus of significance with regard to the general anatomy:
The cornea is continuous with the sclera, which in turn is continuous with the dura.
The choroid, a highly vascular, highly pigmented layer between the sclera and the retina, is continuous with the ciliary body and the iris.
The pigment epithelium is a single cell layer thick, and comes from the outer layer of the original optic cup.

Approximately 80% of the eye's volume is the vitreous, which is a clear medium containing collagen type II, hyaluronic acid, proteoglycans, and a variety of macromolecules including glucose, ascorbic acid, amino acids, and a number of inorganic salts. The overall composition exemplifies a delicate, transparent gel composed of a highly hydrated double network of protein fibrils and charged polysaccharide chains. By weight, vitreous is ˜99% water and 0.9% salts. The remaining 0.1% is divided between protein and polysaccharide components. Most of the protein is found in or associated with 10-20 nm heterotypic collagen fibrils composed of a small collagen type V/XI core wrapped in a thick layer of collagen type II (75% of the fibril by mass). The exterior of each fibril is decorated with covalently bonded collagen type IX and other glycoproteins. Collagen IX contains four short, coiled noncollagenous domains separated by three triple-helical collagenous domains. Two of the collagenous domains are aligned with, and crosslinked to, the axis of the fibrils, but the third strut-like collagenous domain is sterically forced to project out from the fibril by a heparin-sulfate glycosaminoglycan (GAG) chain that is covalently bonded to the adjacent, hinge-like noncollagenous domain. The network of collagen fibrils has been presumed responsible for the mechanical properties of the vitreous because of the load-bearing capacity of collagen and because the vitreous does not fully collapse with the enzymatic removal of hyaluronan. It has been suggested that hyaluronan (HA) polysaccharide chains play a passive role in the vitreous by filling the space between the fibrils to prevent extensive aggregation. Literature indicates that the vitreous shrinks after removal of hyaluronan, and morphologically the collagen network ‘relaxes’ from having relatively straight to significantly curved fibrils.

In the case of severe intraocular inflammation, there may be significant damage to retinal and uveal tissues. The process is initiated by T- and B-lymphocytes, but augmented and maintained by polymorphonuclear leukocytes (PMNs) and macrophages. Chemical mediators, such as arachidonic acid metabolites, proteolytic enzymes, and oxygen metabolites are responsible for the tissue damage evident in ocular inflammatory conditions, such as uveitis (infectious or non-infectious).

With specific reference to the vitreous, inflammatory diseases of various aetiologies produce opacification, liquefaction, and shrinkage. Additional changes include cellular proliferation and transformation leading to fibrosis in cases of prolonged inflammation. In some eyes the fibrosis is primarily cortical while in others it is extensive. Those inflammations with outpouring of a fluid exudate lead to detachment of the vitreous from the posterior eye and extensive shrinkage. In such eyes the vitreous becomes heavily organized and opaque in the central eye behind the lens, obscuring the view of the posterior fundus. In young eyes vitreo-retinal adhesions often form at the sites of inflammation, leading to traction on the retina and ciliary body; retinal tears may result from the traction. Exudate in many inflammatory vitreal inflammations tends to collect at the vitreous base where it organizes into scar tissue. The scar is formed by the retina and ciliary body, but there are also fibrosis-produced monocytes that become transformed into fibroblasts (Hogan, 1975).

It is therefore not surprising that drug delivery to the posterior segment is particularly challenging due to the anatomical and vascular barriers to both local and systemic access.

The device of the present invention can respond to inflammatory molecules, such as the abovementioned chemical mediators, or conditions created within the eye inherent of the infection and/or inflammatory response, that contribute to the pathology of intraocular diseases such as posterior uveitis (which may have an infectious aetiology) and endophthalmitis by effecting polymeric erosion with resultant drug dissolution and release.

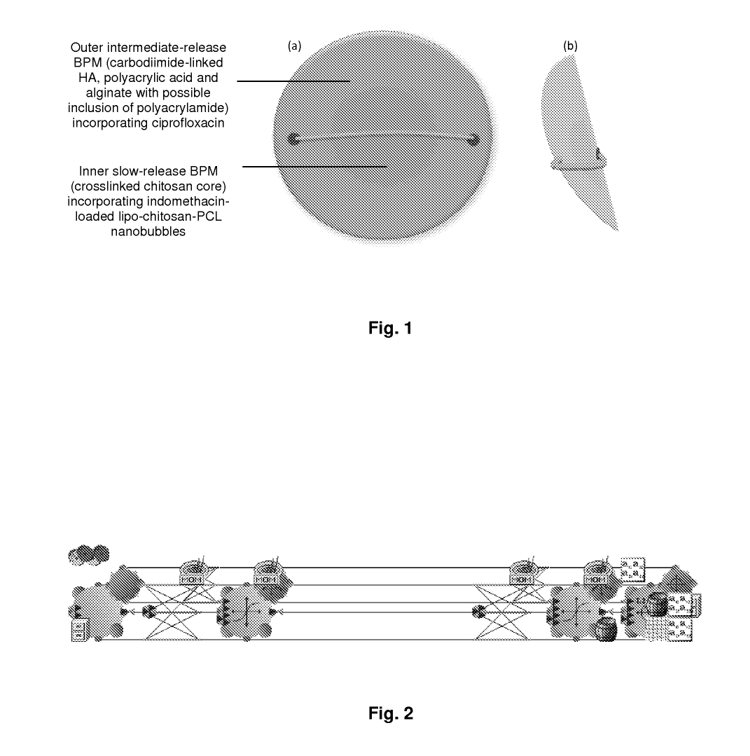

In one embodiment of the device, there is provided a multi-component system incorporating two differential release bioresponsive polymeric matrices (BPMs), an antibiotic and an anti-inflammatory agent-loaded nanosystem (NS) (FIG. 1). The outer BPM was designed for fast to intermediate drug release for the therapeutic management of preliminary inflammation and concurrent infection. The inner crosslinked core matrix, incorporating an indomethacin-loaded nanosystem, was chemically modified to release the nanosystem at a slower rate (delayed release) than the outer matrix for chronic responsive management of the ensuing inflammatory condition. The differential release BPMs were simultaneously originated from polymers susceptible to free radical degradation employing the concept of interpenetrating network formation in the presence of a suitable crosslinking agent.

The BPMs erode and release the anti-inflammatory agent and antibiotic in response to an inflammation-related stimulus, such as the highly reactive intermediates including O²⁻, H₂O₂, chloramines and hydroxyl (OH⁻) radicals that are released from activated leukocytes both in vitro and during acute and chronic intraocular inflammatory reactions in vivo. Thus, release of the anti-inflammatory agent from the bioresponsive device will be individualised and synchronised directly with the needs of the patient as the level of anti-inflammatory agent released from the device will correlate with the level of inflammation experienced by that patient on a particular day. It is anticipated that release of the inflammation reducing agent in this manner could minimise adverse reactions related to the agent.

Several design criteria for the device were devised. These include:
(i) polymer biodegradability and biocompatibility as inherent properties of both the matrix platforms and nanosystem, with minimal propensity to induce inflammation, or uveal or retinal toxicity upon implantation, and sterilisability;
(ii) the capability to act in a bioresponsive manner (e.g. respond to inflammation or other proposed stimulus at the implantation site), and to maintain the drug concentration above therapeutic levels in the vitreous cavity for the duration of the infection and the inflammatory state, and
(iii) easy insertion of the implant device into the desired site (e.g. vitreous cavity) and acceptable size with respect to the anatomy.

The outer BPM can be formed from one or more anionic biopolymers, such as hyaluronic acid, that undergo biologically observed free radical degradation (i.e. inflammation-responsive degradation) However, their rapid degradation (which is also observed in the presence of enzymes such as hyaluronidase) precludes the isolated use of the native polymer for drug delivery. The BPMs can therefore also incorporate alginate, polygalacturonate, or methylcellulose (polyacetals), or poly (ethylene) oxide, or poly (acrylic acids) that are susceptible to free radical induced degradation. Such polymers are conjoined by ether and acetal (i.e. glycosidic linkages). Their monomers are linked wholly or mainly by —C—O—C— bonds and are polyethers or polyacetals. The ratio of alginate:hyaluronic acid is typically about 16:1, and the ratio of alginate:poly (acrylic acid) is typically about 4:1. The proposed mechanism is based on a known disproportionation of ether free radicals, which is induced by hydroxyl radicals (in periodate solutions). Scission of the polymeric chains could occur solely by ether-type disproportionation, or by glycol cleavage following ring opening caused by disproportionation involving the ring oxygen atom. The susceptibility of glycuronans to periodate degradation might be in part due to the known ease of formation of free radicals from alpha-hydroxy acids by abstraction of H-5 followed by ring opening and glycol fission by periodate (Scott and Tigwell, 1973).

The inner (or core) BPM can be formed from cationic low-molecular-weight carbohydrates with which hydroxyl radicals react by abstraction of carbon-bonded hydrogens. Such polymers include, but are not limited to, chitosan. Hydroxyl radicals react with low-molecular-weight carbohydrates by abstraction of carbon-bonded hydrogens, while the reactivity of H atoms is more than an order of magnitude lower. Due to a different reaction geometry present in chitosan, the rate constants of the reactions of OH radicals with polymers are lower than for the low-molecular-weight analogues. They depend on the molecular weight and conformation of the macromolecules and, to a certain extent, also on their concentration.

The inner and outer BPMs can be exposed to chemical crosslinking processes to increase matrix interconnectivity and strength. Matrices can be chemically crosslinked with gluteraldehyde. Carbodiimide coupling can also be instituted to increase the interconnectivity of the matrix. This can be accomplished in the presence of hydroxysuccimide and N,N-dicyclohexylcarbodiimide (DCC) employing aluminium chloride (AlCl₃) as a catalyst for the interpolymeric coupling reaction (Friedel-Crafts acylation). The ratio of chitosan to the gluteraldehyde cross-linking agent in the inner polymeric matrix is typically about 7:1.

The BPMs as described in more detail in the examples below were formulated to meet the following design criteria:
(i) minimal erosion and drug release under in vitro conditions simulating a normal physiological intraocular state;
(ii) enhanced erosion under in vitro conditions simulating a pathological inflammatory intraocular state created in the presence of hydroxyl radicals generated via the Fenton reaction. A steadily rapid modulated erosion of the outer BPM is necessitated for management of the initial infection and acute inflammatory response;
(iii) the inner (core) BPM should display modulated inflammation-responsive erosion at a rate slower than that of the outer layer;
(iv) minimal swellability, expressed as water absorption capacity.

Significantly, the anticipated system can be nano-enabled, comprising crosslinked bioresponsive polymeric matrices (BPMs) incorporating an antibiotic and fixated with a uniformly interspersed nanosystem (NS), such as nanospheres, nanocapsules, nano/microbubbles, nanotubes or nanofibres, as drug reservoirs for inflammatory tissue targeting within the core BPM. The use of nanotechnology-based drug delivery systems prolongs exposure of the drug by controlled release for improved therapeutic efficacy. Nanosytems, when injected into the vitreous, have the propensity to migrate through the retinal layers and tend to accumulate in the retinal pigment epithelium (RPE) cells (Bourges et al., 2003). Thus, inflamed tissues can be specifically targeted. When released within the vitreous from the implant, Sahoo et al. (2008) reported that nanosytems did not induce inflammatory reactions in the retinal tissue, nor was the organisation of the surrounding ocular tissues compromised. The potential of a dispersion of solid nanosystems to dramatically improve a delivery platform's thermal and mechanical integrity was noted by Balazs and Buxton (2004). Furthermore, these systems demonstrate the intrinsic potential to serve as targeted, bioresponsive/self-regulated delivery systems (Li et al., 2005).

The nanosystem can be a polymerically-enhanced lipoid nanosystem. The applicant has shown that such a nanosytem has the following advantages:
(a) incorporation of poorly water-soluble drugs, which is largely independent of the liposome bilayer physicochemical properties;
(b) prolonged lifetime attributed to the polymeric component;
(c) tissue distribution, which will be largely lipid dose independent, such that therapeutic dose escalation produces increasing drug effects with minimal changes in pharmacokinetics; and
(d) the facilitation of the addition of ligands or other functionalities to the polymer surface layer through chemical modifications.

Gas can also be introduced into the nanosystem to create nanobubbles, which possess a reduced density and a purportedly enhanced propensity to migrate though the ocular tissues upon release from the inner BPM.

The pharmaceutically active agent or drug of the outer polymeric matrix is typically an antibiotic, such as ciprofloxacin or other fluoroquinolones (e.g. moxifloxacin, gatifloxacin, levofloxacin), or other suitable antibiotics or antifungal agents (e.g. vancomycin, amikacin, gentamicin, tobramycin, ceftazidime. Amphotericin B) or can be an anti-inflammatory agent. The outer polymeric matrix could even include two pharmaceutically active agents, e.g. an antibiotic and an anti-inflammatory agent. The pharmaceutically active agent of the inner polymeric matrix is typically a steroidal or non-steroidal anti-inflammatory agent, such as the non-steroidal agent, indomethacin.

Lipo-nanobubbles were thus developed which incorporated poly(c-caprolactone) (PCL), having an affinity for inflamed tissue and possessing the potential to penetrate ocular barriers (e.g. the BRB) by an endocytic process, and the mucoadhesive chitosan. The positively charged chitosan is also an ocular barrier permeation-enhancer and additionally prevents nanosystem degradation caused by the adsorption of lysozyme and reduces opsonization and complement activation. Phospholipids were also incorporated in the nanosystem to enhance distribution within the inflamed tissues.

The device can be implanted either intrasclerally, sub-Tenon, on the sclera, or on the pars plana. The device can contain one or more apertures to facilitate suturing at the preferred implantation site, specifically with reference to the pars plana implantation site. The apertures can be created by using a high-powered laser or custom designed tabletting equipment (e.g. a punch set). The aperture(s) can be shaped so that when the polymeric matrix degrades, the surface area of the biodegradable portion of the matrix remains relatively constant. The aperture(s) can be centrally or marginally placed.

The invention will now be described in more detail with reference to the following non-limiting examples which describe an implantable intraocular device for providing inflammation-responsive delivery of an anti-inflammatory agent and antibiotic for the treatment of posterior segment inflammatory disorders. In the examples, indomethacin is used as only one possible example of the anti-inflammatory agent and ciprofloxacin is used as an example of the antibiotic.

### EXAMPLES

### Synthesis of lipo-chitosan-poly(ε-caprolactone) nanobubbles

Poly(ε-caprolactone) (PCL) (20 mg) and an anti-inflammatory agent, indomethacin (20 mg), were dissolved in 5 mL acetone. Phospholipids, disteroyl phophatidylcholine (20 mg) and disteroyl phosphatidylethanolamine (5 mg), were optionally included in the drug-polymer solution, Chitosan (low molecular weight) (40 mg) was dissolved in 15 mL 0.05M HCl. Tween® 80 (0.01 mL) was included as a surfactant for bubble generation. The chitosan solution was slowly added to the phospholipid-PCL-indomethacin solution with sonication for 1 minute under a headspace of air to create gas-filled nanobubbles—gas entrapped within a nanogel shell (20 kHz sonicator, VibraCell, Sonics and Materials, Inc., Danbury, Conn., USA). The organic solvent was subsequently evaporated with gentle stirring for 3 hours. The interaction between the carboxyl or hydroxyl groups of the anionic PCL and the amine groups of chitosan formed immediate polyionic nanogels. Thereafter chitosan (medium molecular weight) (800 mg) was dissolved in the nanobubble suspension to effect further coating of the formed nanogels in a mucoadhesive polysaccharide coating. The stability of the formed nanobubbles was maintained through freezing at −70° C. prior to incorporation as the core of the device. Gas-filled nanobubbles were created through subsequent sonication for 1 minute under a headspace of air.

### Formulation of the Bioresponsive Polymeric Matrices

For the intermediate release outer BPM, a 4% sodium alginate-1% polyacrylic acid (Carbopol 974)-3% hydroxysuccimide-0.25% hyaluronic acid (HA)-2.5% gluteraldehyde-0.25% ciprofloxacin aqueous solution was prepared, instituting carbodiimide coupling chemistry to increase the interconnectivity of the matrix. N,N′-dicyclohexylcarbodiimide (DCC), which is commonly used as a coupling agent, was employed to facilitate coupling between the HA and alginate, and the polyacrylic acid. DCC (300 mg) was dissolved in ethanol and dispersed within the polymeric solution. The ratio of alginate:hyaluronic acid was about 16:1, and the ratio of alginate:poly (acrylic acid) was about 4:1

For the inner (core) BPM, the chitosan solution incorporating the lipo-chitosan-PCL nanobubbles was prepared as described above. The anionic polymer-drug solution (0.3 mL) was distributed to plastic moulds containing 0.05 mL of an acidified 3% AlCl₃ solution, where the AlCl₃ serves as a catalyst for the interpolymeric coupling reaction (Friedel-Crafts acylation). The cationic polymer solution (0.1 mL) was added to the centre of the mould. Diffusional development of two separate interpenetrating networks, and simultaneous curing of the chitosan core and outer matrix, was allowed to occur over 12 hours.

The final implants were allowed to dry for 48 hours under reduced pressure at 25° C. Once dried, one or more apertures may optionally be created employing, for example, a high-powered laser system or a specially designed punch set.

### Experimental Design for the Bioresponsive Polymeric Matrices for Selection of Pertinent Variables Impacting the Formulation Process

Preliminary investigations were undertaken for the identification of critical formulatory components and their upper and lower desirable levels. The fixed ratio presence of alginate:poly acrylic acid was found to be integral for establishment of the outer matrix. The specified ratio of chitosan to the gluteraldehyde crosslinking agent (˜7:1) also proved essential for the formation of a robust inner matrix.

Factors were selected that would ultimately impact on the responses displayed by the preliminary system. Optimization of the intraocular implant was conducted by constructing and analysing a four-factor, three-level (3⁴) Box-Behnken statistical design on MINITAB®, (V15, Minitab, USA) as depicted in Table 1.

TABLE 1
Factors and levels of independent variables generated
by the 3⁴ Box-Behnken Design

| | Hyaluronic Acid | Hydroxysuccimide | DCC | AlCl₃ |
|---|---|---|---|---|
| Formulation | (% w/v) | (% w/v) | (% w/v) | (% w/v) |
| 1 | 0.25 | 3 | 8 | 8 |
| 2 | 0.50 | 4 | 6 | 6 |
| 3 | 0.25 | 4 | 6 | 8 |
| 4 | 0.25 | 3 | 6 | 6 |
| 5 | 0.25 | 3 | 4 | 8 |
| 6 | 0.00 | 2 | 6 | 6 |
| 7 | 0.25 | 3 | 8 | 4 |
| 8 | 0.00 | 3 | 6 | 4 |
| 9 | 0.25 | 2 | 6 | 6 |
| 10 | 0.25 | 4 | 6 | 4 |
| 11 | 0.50 | 2 | 6 | 6 |
| 12 | 0.25 | 2 | 6 | 8 |
| 13 | 0.00 | 3 | 4 | 6 |
| 14 | 0.50 | 3 | 6 | 8 |
| 15 | 0.25 | 3 | 6 | 6 |
| 16 | 0.00 | 4 | 6 | 6 |
| 17 | 0.50 | 3 | 6 | 4 |
| 18 | 0.25 | 4 | 4 | 6 |
| 19 | 0.25 | 2 | 4 | 6 |
| 20 | 0.25 | 2 | 6 | 4 |
| 21 | 0.00 | 3 | 6 | 8 |
| 22 | 0.00 | 3 | 8 | 6 |
| 23 | 0.25 | 3 | 6 | 6 |
| 24 | 0.50 | 3 | 8 | 6 |
| 25 | 0.25 | 4 | 8 | 6 |
| 26 | 0.50 | 3 | 4 | 6 |
| 27 | 0.25 | 3 | 4 | 4 |

Evaluation of the In Vitro Bioresponsive Drug Release Behaviour from the Experimental Design-Derived Bioresponsive Polymeric Matrices

In the selection of inflammation as a stimulus, the in vitro degradation of the crosslinked BPMs by varying levels of chemical inflammatory mediators (hydroxyl radicals) generated via the Fenton reaction (Equation 1) was examined:

Fe²⁺+H₂O₂→Fe³⁺+OH.+OH⁻ [Equation 1]

A modified closed-compartment USP 31 dissolution testing apparatus was used. Each accurately weighed device (separately loaded with indomethacin in the core BPM or ciprofloxacin in the outer BPM) was either exposed to normal conditions (N) following immersion in 4 mL SVH (comprising phosphate-buffered saline with 0.03% ^{v}/ᵥ hyaluronic acid, 37° C.) at physiological pH (7.4), or pathological inflammatory conditions (F) in 4 mL SVH containing 0.05M Fenton's reagent. Briefly, each accurately weighed BPM was placed in SVH which contained 1 mL 0.1M FeSO₄. Complex formation between Fe²⁺ and the polymeric chains comprising the BPM was initiated and permitted to proceed for 1 hour after which 1 mL 0.1M H₂O₂ was added, thus generating 100 μmol of hydroxyl radicals. This fell within the range of hydroxyl radicals reportedly generated during pathological inflammatory states (Yiu et al., 1992). The drug release was thus reported at normal physiological and pathological conditions to enable assessment of an inflammation responsive mode of degradation would be facilitated.

The samples were placed in closed vials and placed in an oscillating laboratory incubator (Labcon® FSIE-SPO 8-35, California, USA), set to 20 rpm. Balancing withdrawal of samples was undertaken at 3, 7, 14, 21 and 28 days. All aliquots withdrawn were subjected to filtration (0.22 μm PVDF, Millipore Corporation, Bedford, Mass., USA) and appropriately diluted prior to spectrophotometric analysis at the λₘₐₓ for indomethacin (318 nm) and ciprofloxacin (278 nm) in SVH. The componential polymeric absorbance of the device, together with the influence of the Fenton's reagent on the absorbance readings at the respective wavelengths were taken into account. All analyses were conducted in triplicate (n=3). A model-independent approach was used to compare the dissolution data for both the inner and outer BPM for ciprofloxacin and indomethacin release. For this purpose a mean dissolution time at 28 days (MDT) was calculated for each formulation, defined as the sum of different release fraction periods obtained for dissolution studies endured in SVH, divided by the initial loading dose (Pillay and Fassihi, 1998) as exemplified in Equation 2:

$$MDT = {\sum }_{i = 1}^{n} ti \frac{{M}_{i}}{{M}_{∞}} \qquad [ Equation 2 ]$$

where Mₜ is the fraction of dose released in time ti=(tᵢ+tᵢ₋₁)/2 and M∞ corresponds to the loading dose and a maximum MDT refers to the fastest drug release achievable (Govender et al., 2005).

### Investigation of the Transitional Micromechanical Behaviour and Fluid Uptake of the Experimental Design-Derived Intraocular Implants

All 27 formulations (containing both ciprofloxacin and indomethacin) were exposed to both normal (N) and pathological (F) testing conditions as described for in vitro drug release evaluation. At each time point (0, 3, 7 14, 21, and 28 days) the implant was removed from the simulated physiological fluid, excess liquid blotted with filter paper, and the water absorption capacity and textural attributes evaluated in triplicate. The hydrated implant was weighed at each time point to assess the swollen weight as an indication of the water absorption capacity (WAC) as follows:

$$WAC ( \% ) = \frac{{W}_{s} - {W}_{d}}{{W}_{d}} \times 100 \qquad [ Equation 3 ]$$

where Wₛ is the swollen weight and W_{d} is the dry weight of the respective BPM.

The physicomechanical properties were assessed through textural profiling of the device using a calibrated Texture Analyser (TA.XT.plus Texture Analyser, Stable Microsystems®, Surrey, UK) fitted with a 5 kg load cell was employed for determination of the matrix hardness (N/mm, calculated as the gradient of the force-displacement profile during the compression phase) and deformation energy (N.m or J, calculated as the area under the force-displacement curve, AUC) of unhydrated BPMs and the bioadhesive matrix, using a 2 mm flat-tipped steel probe, and matrix resilience of unhydrated and SVH-hydrated BPMs and the bioadhesive matrix, using a 36 mm cylindrical steel probe. The settings for analysis are highlighted in Table 2.

TABLE 2
Textural parameters for determination of matrix hardness,
deformation energy and matrix resilience

| | Matrix hardness and | Matrix resilience |
|---|---|---|
| Parameter | deformation energy settings | settings |
| Pre-test speed | 1.00 mm/s | 1.00 mm/s |
| Test speed | 2.00 mm/s | 2.00 mm/s |
| Post-test speed | 10.0 mm/s | 10.0 mm/s |
| Target mode | Force | 10% strain |
| Target force | 0.98067N | — |
| Trigger type | Auto (force) | Auto (force) |
| Trigger force | 0.04903N | 0.04903N |
| Load cell | 5 kg | 5 kg |

### Optimization of the Formulatory Components

Following generation of the polynomial equations relating the dependent and independent variables, the formulation process was optimised under constrained conditions for the measured responses, which were:
MDT of indomethacin at 28 days under normal and pathological conditions (MDT I N and F, respectively)
MDT of ciprofloxacin at 28 days under normal and pathological conditions (MDT C N and F, respectively)
Change/difference in the MDT of indomethacin from normal to pathological conditions (Δ MDT I)
Change/difference in the MDT of ciprofloxacin from normal to pathological conditions (Δ MDT C)
Rate of change in water absorption capacity under normal and pathological conditions (Δ WAC N and Δ WAC F, respectively)
Overall rate of change in normalised textural properties i.e. averaged rate of change in resilience, hardness and deformation energy under normal pathological conditions (Δ Textural properties N and F, respectively)
Rate of change in each textural attribute (resilience, hardness, and deformation energy) under normal and pathological conditions (Δ Resilience N and F, Δ Hardness N and F, Δ Deformation Energy N and F)

Simultaneous equation solving for optimization of the formulation process was performed to obtain the levels of independent variables, which would exemplify the bioresponsive capabilities of the device. i.e. maximization of the ΔMDT, and minimization of the WAC, such that the device would release negligible drug under normal conditions but release increased levels of drug on exposure to an inflammatory stimulus, and swell minimally on exposure to the physiological fluids of the eye.

### Concurrent Optimization by ANN for Statistical Validation

Concurrent optimization was conducted by employing the feedback Multilayer Perceptron (MLP) neural network to train the empirical input Δ MDT I data with static back propagation. FIG. 2 illustrates the typical construction of the MLP network. The input data (obtained from the comparative drug release investigations under normal and pathological conditions) were trained. The main advantage of these networks is that they can approximate any input/output map.

A genetic algorithm with a Sigmoid Axon transfer function and Conjugated Gradient learning rule was employed for the hidden input and output layers. A maximum of 10,000 epochs were run on NeuroSolutions Version 5.0 (NeuroDimension Inc., Gainsville, Fla.) for ensuring optimal training of data.

Kinetic Analysis of Drug Release from the Optimum Formulation

To analyze the in vitro release data of the optimum formulation various kinetic models were used to describe the release kinetics. The zero order rate equation (Equation 4) describes the systems where the drug release rate is independent of its concentration (Hadjiioannou at al., 1993). The first order equation (Equation 5) describes the release from a system where release rate is concentration dependent (Bourne, 2002). Higuchi (1963) described the release of drugs from an insoluble matrix as a square root of time dependent process based on Fickian diffusion (Equation 6). The Hixson-Crowell cube root law (Equation 7) describes the release from systems where there is a change in surface area and diameter of particles or tablets (Hixson and Crowell, 1931).

C=k₀t [Equation 4]

where, k₀ is the zero-order rate constant expressed in units of concentration/time and t is the time.

Log C=Log C₀−kt/2.303 [Equation 5]

where, C₀ is the initial concentration of drug and k is the first order constant.

Q=Kt^{1/2} [Equation 6]

where, K is the constant reflecting the design variables of the system.

Q₀^{1/3}−Qₜ^{1/3}=K_{HC}t [Equation 7]

where, Qₜ is the amount of drug released in time t, Q₀ is the initial amount of the drug in tablet and K_{HC} is the rate constant for Hixson-Crowell rate equation.

The following plots were made: cumulative % drug release vs. time (zero order kinetic model); log cumulative of % drug remaining vs. time (first order kinetic model); cumulative % drug release vs. square root of time (higuchi model) log cumulative % drug release vs. log time (korsmeyer model) and cube root of drug % remaining in matrix vs. time (hixson-crowell cube root law).

### Mechanism of Drug Release

Korsmeyer et al. (1983) derived a simple relationship which described drug release from a polymeric system (Equation 8). To postulate the mechanism of drug release, the drug release data (generally less than 60%) was fitted in Korsmeyer-Peppas model:

Mt/M_{∞}=Ktⁿ [Equation 8]

where Mt/M_{∞} is fraction of drug released at time t, K is the rate constant and n is the release exponent. The n value is used to characterize different release mechanisms as for cylindrical shaped matrices, which may be n=0.45 for Fickian diffusion, 0.45<n<0.89 for anomalous (non-Fickian) diffusion, n=0.89 for case-II transport, and n>0.89 for Super case-II transport

### Componential Physicochemical Evaluation of the Device

### Scanning Electron Microscopy on Lipo-Chitosan-PCL Nanobubbles

Surface morphology of dried nanobubbles incorporated within the core BPM was evaluated on a JEOL 840 SEM (JEOL, Japan) to view the overall and in-depth surface architecture to qualitatively elucidate factors such as shape, size, and degree of aggregation.

### Zeta Potential and Size Analysis of Chitosan-PCL Nanogels

Nanobubble stability was evaluated via zeta potential value determination—a high absolute zeta potential value indicating a high electric charge on the NS surface. Zeta potential was measured employing a Zetasizer Nano ZS (Malvern Instruments Ltd. UK). Size analysis was undertaken using multimodal analysis at a scattering angle of 90° and temperature of 25° C. The hydrodynamic particle size will be calculated as the value of z-average size±SD. The width of the size distribution is indicated by the polydispersity index (PI).

### Fourier-Transform Infrared Analysis of the Device

The vibrational molecular transitions of the nanobubbles incorporated within the inner crosslinked core, and the outer matrix in comparison with the native system components were characterized for the attainment of important microstructural information via their Fourier-transform infrared (FTIR) spectra, recorded on a PerkinElmer® Spectrum 100 Series fitted with a universal ATR Polarization Accessory (PerkinElmer Ltd. Beaconsfield, UK). Spectra were recorded over the range 4000-25 cm⁻¹, with a resolution of 4 cm⁻¹ and 32 accumulations.

### Results

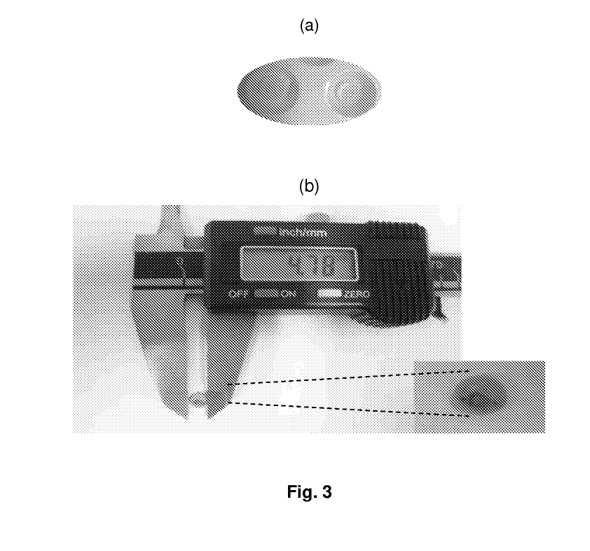

The simultaneous formation of the multi-crosslinked BPMs culminating in the final device in polyethylene moulds of appropriate curvature is highlighted in FIG. 3, as well as the diameter of the dried implant. There was dramatic shrinkage of the hydrogels implicated in implant formation due to a potentially high degree of crosslinking with resultant enhanced interconnectivity of the component polymers.

The drug release profiles generated for the experimentally-derived formulations attest to the bioresponsive potential of the implants, as in general, a higher degree of drug release was achieved when implants were exposed to pathological conditions (hydroxyl radicals generated by Fenton's reaction). In vitro levels of ciprofloxacin attained were above the MIC₉₀ of common pathogens for ciprofloxacin (>0.8 μg/mL, refer to Table 3) being >2 μg/mL and >10 μg/mL in all cases, under normal and pathological conditions, respectively. Intraocular levels achieved following topical application of ciprofloxacin was demonstrated by Yagci et al. (2007). Following infection with an intravitreal inoculum of Staphylococcus aureus in New Zealand Albino Rabbits, the efficacy of topical ciprofloxacin was evaluated 24 h after the inoculation, and compared to topical application in normal eyes. In the normal and inflamed eyes, mean aqueous concentrations of ciprofloxacin were 2.16 and 3.65 μg/mL. Mean vitreous concentrations of ciprofloxacin were 0.08 and 0.32 μg/mL, in normal and inflamed eyes, respectively. This highlights the potential of this system to deliver adequate drug levels intraocularly.

TABLE 3
MIC₉₀ of common ocular pathogens for
ciprofloxacin (adapted from Yegci et al., 2007)

| Bacterial Species | MIC₉₀ (μg/ml) |
|---|---|
| Escherichia coli | 0.02; 0.083 |
| Enterobacter | 0.206 |
| Klebsiella | 0.295 |
| Proteus | 0.267 |
| Pseudomonas | 0.50; 0.626 |
| Haemophilus influenzae | 0.014 |
| Staphylococcus aureus | 0.57; 0.796 |
| Staphylococcus epidermidis | 0.25; 0.375 |
| Streptococcus pyogenes | 0.782 |
| Propionibacterium acnes | 0.35 |
| Bacillus cereus | 0.25 |
| Serratia | 0.12 |

*Cases where two values are quoted indicate differences in results obtained by investigators

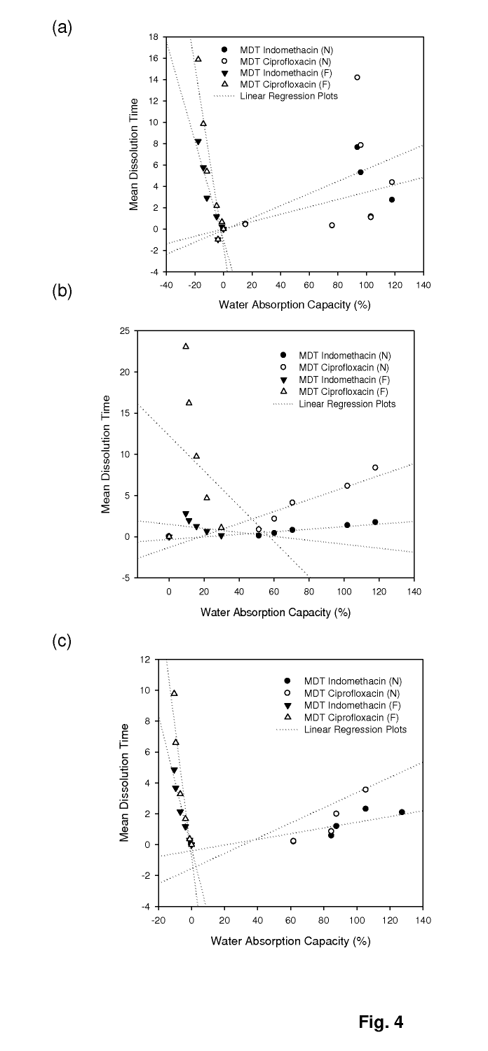

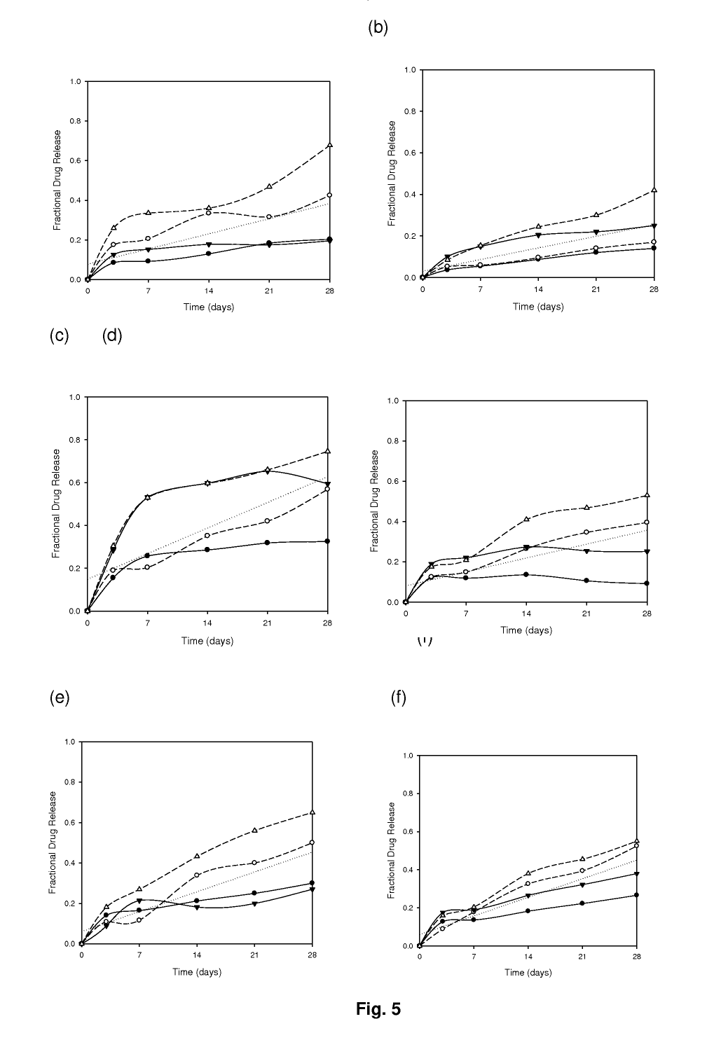

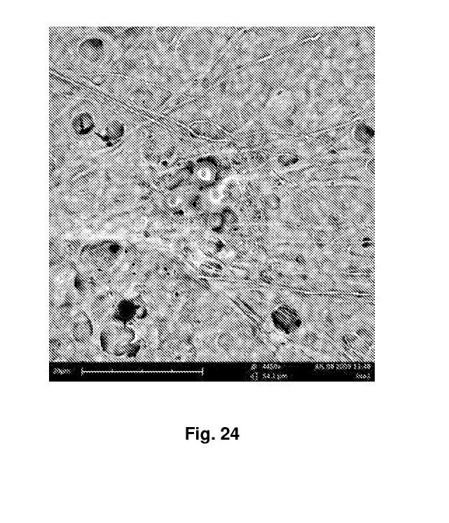

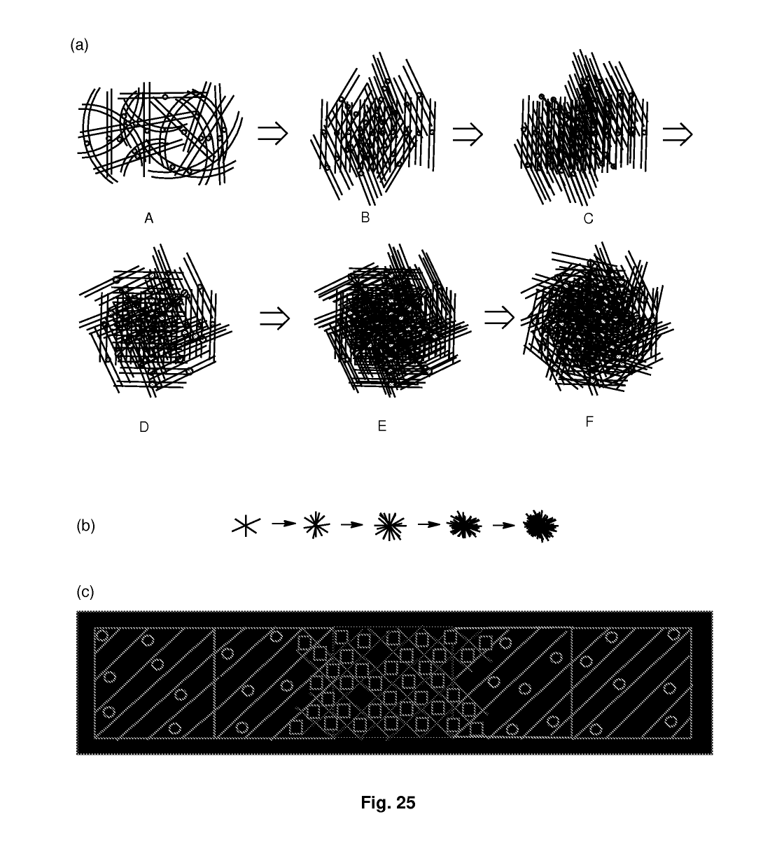

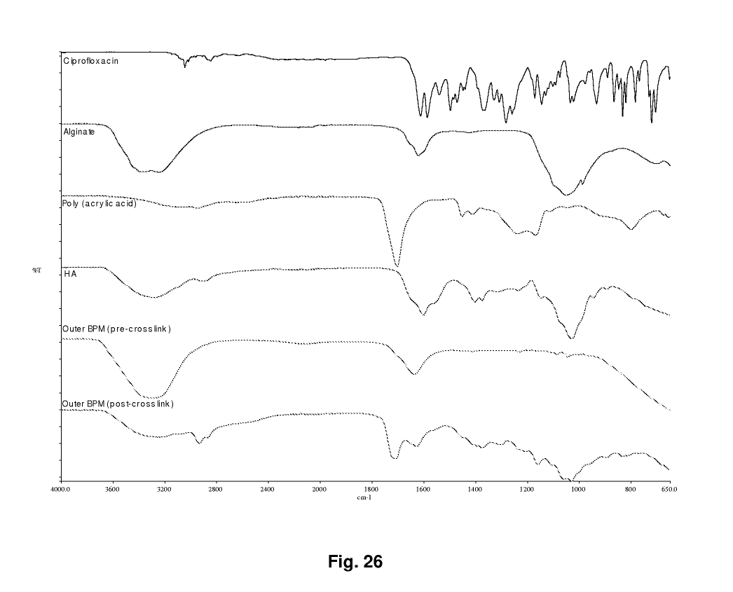

Various degrees of bioresponsiveness were attained for the experimentally-derived devices, with ΔMDT of Indomethacin ranging from 0-32.606. For ciprofloxacin the ΔMDT ranged from 5.109-25.956 Diverse correlatory relationships were derived between the MDT and WAC of the formulations under normal and inflammatory conditions (FIG. 4, Table 4); an indication of the differing types and degrees of crosslinking attained through variation of the formulatory components. The measured responses for all experimentally-derived formulations are provided in Table 5. Drug release profiles clearly highlight that for the majority of formulations, there was enhanced release of both indomethacin and ciprofloxacin from the matrices when exposed to inflammatory conditions (FIGS. 5-8). There was a strong positive correlation between the MDT of both indomethacin and ciprofloxacin with the WAC of the device. The transitions in the textural properties of the formulations with time are depicted in FIGS. 9-12

TABLE 4
The relationship between the mean dissolution
time and fluid imbibement of formuations

| | Correlation coefficient (R²) for MDT vs. WAC | | | |
|---|---|---|---|---|
| Formu- | Indomethacin | Cipro- | Indomethacin | Cipro- |
| lation | (N) | floxacin (N) | (F) | floxacin (F) |
| 1 | 0.1856 | 0.1782 | −0.2364 | −0.2608 |
| 2 | 0.7647 | 0.7721 | −0.8961 | −0.9025 |
| 3 | 0.7621 | 0.6714 | −0.1731 | −0.1904 |
| 4 | 0.2688 | 0.1215 | −0.3015 | −0.3416 |
| 5 | 0.8953 | 0.8790 | −0.9597 | −0.9753 |
| 6 | 0.7845 | 0.7562 | −0.6888 | −0.6932 |
| 7 | 0.7844 | 0.7803 | −0.4481 | −0.4547 |
| 8 | 0.7776 | 0.7001 | −0.7547 | −0.7704 |
| 9 | 0.6009 | 0.5467 | −0.6643 | −0.6705 |
| 10 | 0.4672 | 0.4090 | −0.9603 | −0.9514 |
| 11 | 0.7370 | 0.7124 | −0.4827 | −0.5278 |
| 12 | 0.1819 | 0.2326 | −0.8202 | −0.8733 |
| 13 | 0.3681 | 0.3745 | −0.8365 | −0.8278 |
| 14 | 0.7523 | 0.7897 | −0.1958 | −0.1926 |
| 15 | 0.2688 | 0.1215 | −0.3015 | −0.3416 |
| 16 | 0.8972 | 0.8956 | −0.4670 | −0.5024 |
| 17 | 0.7199 | 0.7148 | −0.5144 | −0.5276 |
| 18 | 0.7118 | 0.6998 | −0.1809 | −0.2239 |
| 19 | 0.7890 | 0.7569 | −0.7033 | −0.6922 |
| 20 | 0.9243 | 0.9368 | −0.2254 | −0.2423 |
| 21 | 0.9061 | 0.8060 | −0.6416 | −0.6604 |
| 22 | 0.6045 | 0.5866 | −0.1132 | −0.1204 |
| 23 | 0.2688 | 0.1215 | −0.3015 | −0.3416 |
| 24 | 0.8378 | 0.7918 | −0.9787 | −0.9497 |
| 25 | 0.8018 | 0.8197 | −0.5375 | −0.5481 |
| 26 | 0.8466 | 0.6887 | −0.8913 | −0.8870 |
| 27 | 0.6577 | 0.5568 | −0.3867 | −0.3981 |

### Response Surface Analysis of the Box-Behnken Design

Factors having notable or significant effects on investigated responses have been further elaborated on to highlight the intricate relationship between the formulatory components of the resultant experimentally-derived formulations. The MDT I N, MDT I F, Δ MDT I, Δ WAC N, and Δ WAC F as measured responses for the experimentally synthesized formulations were included in the statistical design for identification of a formulation with an optimal bioresponsive potential.

Residual analysis (run order, predicted values) for the significant responses of the response surface design data (FIG. 13) generally showed random scatter i.e. no trends, indicating none of the underlying assumptions of the multiple regression analysis were grossly violated. However, some fanning and an outlier was observed for MDT I N (FIG. 13a), indicative of a degree of nonconstant variance. The normal probability plots of the residuals fell on a straight line indicating the data to be normally distributed with no evidence of unidentified variables.

The residuals and standardised residuals indicated that the majority of cases were adequately fitted by the response surface model. Cook's distance was interpreted an overall measure of the combined impact of each observation on the fitted values and considers whether an observation is unusual with respect to both x- and y-values. Unusual observations generated by the model were minimal. The significance of the ratio of mean square variation due to regression and residual error was tested using ANOVA. The theoretical (predicted) values and observed (experimental) values were in fairly close agreement for MDT I N (R²=0.8516), MDT I F (R²=0.8368), Δ MDT I (R²=0.8039), Δ WAC N (R²=0.8476), Δ WAC F (R²=0.7237), respectively, thus indicating the applicability of the regression models and usefulness of response surface plots.

TABLE 5
Measured responses for the device formulations

| | MDT | MDT | MDT | MDT | Δ | Δ | Δ Textural | Δ | Δ | Δ |
|---|---|---|---|---|---|---|---|---|---|---|
| Form | I N | I F | C N | C F | MDT I | MDT C | Properties | WAC N | WAC F | Resilience N |
| 1 | 6.77 | 7.99 | 13.02 | 18.99 | 1.22 | 5.97 | 1.60E−02 | 8.86 | −0.06 | −1.09E−01 |
| 2 | 10.17 | 20.74 | 10.70 | 30.65 | 10.57 | 19.95 | 3.62E−03 | 30.26 | −1.75 | −8.51E−02 |
| 3 | 18.01 | 26.23 | 35.34 | 39.16 | 8.21 | 3.82 | 1.11E−02 | 65.97 | −0.30 | −1.16E−01 |
| 4 | 6.33 | 19.46 | 14.90 | 26.78 | 13.13 | 11.88 | 8.18E−03 | 10.96 | −0.74 | −3.41E−02 |
| 5 | 14.99 | 23.55 | 13.24 | 31.89 | 8.55 | 18.65 | 5.66E−03 | 15.72 | −2.07 | −2.50E−02 |
| 6 | 13.20 | 24.18 | 18.94 | 26.69 | 10.98 | 7.75 | 2.01E−02 | 51.46 | −2.64 | −5.03E−02 |
| 7 | 15.23 | 29.32 | 14.03 | 35.09 | 14.09 | 21.06 | 1.61E−02 | 22.64 | −1.67 | −8.07E−02 |
| 8 | 12.56 | 22.99 | 9.44 | 19.14 | 10.43 | 9.71 | 5.52E−03 | 19.48 | −6.32 | −8.88E−02 |
| 9 | 5.75 | 5.57 | 12.85 | 17.28 | −0.17 | 4.43 | 1.35E−02 | 53.30 | −2.62 | −5.44E−02 |
| 10 | 17.10 | 27.73 | 18.32 | 33.95 | 10.63 | 15.63 | 2.60E−03 | 15.56 | −3.83 | −5.08E−02 |
| 11 | 9.08 | 19.59 | 8.13 | 25.21 | 10.51 | 17.08 | 1.25E−02 | 46.85 | −1.01 | −2.17E−02 |
| 12 | 45.30 | 48.65 | 42.50 | 50.36 | 3.35 | 7.87 | 5.90E−03 | 8.12 | −2.73 | −3.23E−02 |
| 13 | 12.21 | 21.01 | 7.74 | 20.93 | 8.80 | 13.19 | 5.20E−03 | 10.85 | −2.01 | −6.48E−02 |
| 14 | 6.15 | 8.80 | 26.35 | 39.90 | 2.65 | 13.55 | 1.51E−02 | 65.25 | 0.37 | −2.58E−02 |
| 15 | 6.33 | 19.46 | 14.90 | 26.78 | 13.13 | 11.88 | 8.18E−03 | 10.96 | −0.74 | −3.41E−02 |
| 16 | 38.82 | 43.31 | 45.20 | 56.16 | 4.49 | 10.96 | 5.60E−03 | 68.75 | −0.69 | −3.04E−02 |
| 17 | 7.91 | 11.08 | 28.85 | 54.80 | 3.18 | 25.96 | 1.70E−02 | 35.13 | −1.05 | −9.50E−03 |
| 18 | 34.18 | 46.25 | 28.85 | 53.27 | 12.07 | 24.42 | 1.50E−02 | 48.47 | −0.02 | −6.00E−02 |
| 19 | 13.34 | 21.43 | 11.67 | 29.86 | 8.09 | 18.19 | 5.07E−03 | 16.03 | −1.97 | −2.25E−02 |
| 20 | 4.31 | 6.89 | 21.51 | 54.71 | 2.57 | 33.20 | 1.20E−02 | 21.50 | −0.39 | −8.30E−03 |
| 21 | 12.47 | 38.80 | 13.40 | 29.67 | 26.33 | 16.27 | 1.05E−02 | 23.24 | −1.08 | −4.43E−02 |
| 22 | 11.56 | 44.17 | 53.17 | 58.28 | 32.61 | 5.11 | 1.14E−02 | 30.89 | 0.22 | −2.32E−02 |
| 23 | 6.33 | 19.46 | 14.90 | 26.78 | 13.13 | 11.88 | 8.18E−03 | 10.96 | −0.74 | −3.41E−02 |
| 24 | 6.31 | 13.78 | 12.14 | 21.70 | 7.47 | 9.56 | 3.14E−03 | 22.05 | −2.31 | −7.10E−03 |
| 25 | 32.10 | 48.72 | 21.56 | 34.42 | 16.62 | 12.86 | 1.90E−02 | 52.84 | −1.99 | −1.06E−01 |
| 26 | 10.41 | 21.29 | 13.37 | 34.57 | 10.88 | 21.19 | 9.65E−03 | 44.54 | −2.86 | −4.18E−02 |
| 27 | 17.75 | 23.07 | 13.17 | 29.96 | 5.32 | 16.79 | 5.47E−03 | 11.63 | −1.0037 | −3.29E−02 |

| | | Δ | | | Δ |
|---|---|---|---|---|---|
| | Δ | Deformation | Δ | Δ | Deformation |
| Form | Hardness N | Energy N | Resilience F | Hardness F | Energy F |
| 1 | −1.27E−01 | −1.04E−01 | −5.73E−02 | −1.30E−01 | −1.14E−01 |
| 2 | −1.16E−02 | −2.22E−02 | 3.30E−03 | −9.00E−03 | −1.70E−03 |
| 3 | −1.06E−01 | −1.58E−02 | −1.06E−01 | −1.30E−01 | −6.00E−04 |
| 4 | −7.44E−02 | −7.52E−02 | 3.10E−03 | −7.89E−02 | −8.08E−02 |
| 5 | −5.12E−02 | −3.14E−02 | 4.00E−03 | −3.56E−02 | −2.87E−02 |
| 6 | −1.40E−01 | −9.26E−02 | −7.03E−02 | −2.05E−01 | −1.89E−01 |
| 7 | −1.24E−01 | −1.33E−01 | −1.84E−02 | −1.25E−01 | −1.44E−01 |
| 8 | −2.97E−02 | 3.20E−03 | −2.58E−02 | −2.49E−02 | −2.59E−02 |
| 9 | −6.30E−02 | −2.63E−02 | −1.03E−01 | −1.07E−01 | −1.25E−01 |
| 10 | −1.56E−02 | −1.60E−03 | −2.50E−03 | 1.90E−03 | 2.70E−03 |
| 11 | −1.25E−01 | −1.07E−01 | −4.10E−03 | −1.14E−01 | −7.38E−02 |
| 12 | −4.37E−02 | −1.42E−02 | −7.50E−03 | −5.46E−02 | −4.09E−02 |
| 13 | −3.08E−02 | −1.23E−02 | −2.67E−02 | −3.04E−02 | −2.10E−02 |
| 14 | −1.42E−01 | −1.22E−01 | −1.72E−02 | −1.47E−01 | −1.45E−01 |
| 15 | −7.44E−02 | −7.52E−02 | 3.10E−03 | −7.89E−02 | −8.08E−02 |
| 16 | −7.77E−02 | −3.94E−02 | 1.02E−02 | −5.48E−02 | −4.11E−02 |
| 17 | −1.56E−01 | −1.20E−01 | −1.26E−02 | −1.73E−01 | −1.41E−01 |
| 18 | −1.34E−01 | −1.18E−01 | 1.66E−02 | −1.46E−01 | −1.50E−01 |
| 19 | −4.62E−02 | −3.62E−02 | 4.20E−03 | −3.17E−02 | −2.93E−02 |
| 20 | −1.11E−01 | −1.12E−01 | −1.41E−02 | −1.07E−01 | −1.24E−01 |
| 21 | −1.08E−01 | −8.14E−02 | 2.70E−03 | −7.69E−02 | −6.81E−02 |
| 22 | −9.75E−02 | −7.23E−02 | 3.16E−02 | −1.29E−01 | −1.24E−01 |
| 23 | −7.44E−02 | −7.52E−02 | 3.10E−03 | −7.89E−02 | −8.08E−02 |
| 24 | −3.27E−02 | −2.27E−02 | −2.40E−03 | −3.36E−02 | −2.20E−02 |
| 25 | −1.31E−01 | −1.15E−01 | −1.25E−01 | −1.32E−01 | −1.29E−01 |
| 26 | −8.35E−02 | −3.32E−02 | −2.50E−03 | −1.02E−01 | −1.09E−01 |
| 27 | −6.28E−02 | −5.03E−02 | 1.30E−02 | −4.34E−02 | −4.56E−02 |

The Pearson correlation coefficient (R and R-adjusted) represents the proportion of variation in the response that is explained by the model. The R² and R²-adjusted values for the MDT I N, MDT I F, Δ MDT I, Δ WAC N, Δ WAC F models were satisfactory.

The significance of linear and higher-order interaction terms is depicted by the p-values in Table 6.

TABLE 6
Estimated p-values for the measured responses

| | p-value | | | | | |
|---|---|---|---|---|---|---|
| Term (% w/v) | MDT IN | MDT I F | Δ MDT I | Δ WAC N | Δ WAC F | Δ Resilience F |
| HA | 0.4880 | 0.192 | 0.239 | 0.901 | 0.078 | 0.320 |
| NHS | 0.336 | 0.689 | 0.447 | 0.023 | 0.952 | 0.047 |
| DCC | 0.461 | 0.775 | 0.541 | 0.588 | 0.821 | 0.400 |
| AlCl3 | 0.612 | 0.116 | 0.065 | 0.454 | 0.720 | 0.252 |
| HA*HA | 0.993 | 0.780 | 0.656 | 0.021 | 0.347 | 0.762 |
| NHS*NHS | 0.014 | 0.120 | 0.177 | 0.007 | 0.471 | 0.113 |
| AlCl³*AlCl₃ | 0.392 | 0.903 | 0.159 | 0.910 | 0.449 | 0.560 |
| HA*NHS | 0.203 | 0.400 | 0.610 | 0.293 | 0.371 | 0.385 |
| HA*DCC | 0.853 | 0.163 | 0.050 | 0.192 | 0.575 | 0.487 |
| HA*AlCl3 | 0.929 | 0.398 | 0.213 | 0.408 | 0.213 | 0.690 |
| NHS*DCC | 0.768 | 0.392 | 0.325 | 0.306 | 0.656 | 0.677 |
| NHS*AlCl₃ | 0.048 | 0.058 | 0.803 | 0.060 | 0.066 | 0.200 |

In fabricating a bioresponsive device, it is imperative that these features are implicitly accentuated via the drug release behaviour. A low MDT for the drugs from the BPMs is favoured when the implant is exposed to normal physiological conditions. The interaction between NHS and AlCl₃ had a significant effect on the MDT of indomethacin (p=0.048) (FIG. 14a). With DCC serving as the activator, activating the HA towards amide formation with alginate, NHS as the reagent, and AlCl₃ as the catalyst; a stoichiometric ratio of these components is required for optimal crosslinking. Crosslinking is best promoted at lower concentrations of NHS and AlCl₃ (FIG. 16a). An enhanced degree of crosslinking within the outer BPM, forms an intact structure around the inner BPM, retarding swelling and subsequent erosion of both the inner and outer BPMs and subsequent nanosystem release.

A similar result was seen for the MDT of indomethacin when exposed to inflammatory conditions. The interaction between NHS and AlCl₃ had a notable effect on the MDT of indomethacin (p=0.058) (FIG. 14b). Crosslinking of the outer BPM was optimal at lower concentrations of NHS and AlCl₃ (FIG. 16b). An enhanced degree of crosslinking within the outer BPM, forms an intact structure around the inner BPM, retarding swelling and subsequent erosion of both the inner and outer BPMs and subsequent nanosystem release.

A large difference in the MDT of the drug from the device is preferable as the aim is to achieve is system which is inherently bioresponsive, releasing more drug when exposed to pathological conditions. AlCl₃ had a notable effect (p=0.065) on Δ MDT of indomethacin from normal to pathological conditions (FIG. 14c). The difference in MDT was highest at median levels of the catalyst (FIG. 16c). The potential of the catalyst to promote intermolecular and interpolymeric crosslinking was optimal at this level indicating a stoichiometrically sound molar presence of the catalyst in relation to the activator, reagent, and polymers employed. The interaction between the inflammation-responsive HA and DCC emanated in a significant effect on the change in MDT (p=0.050) (FIG. 14c). The MDT was lowest when either correspondingly high levels or low levels of the DCC activators and bioresponsive HA were instituted (FIG. 16c), indicating once again the dependency of origination of an optimally crosslinked BPM on the stoichiometric implementation of components.

A low WAC is an indication of the degree of crosslinking achieved within the implant and is the most favourable situation for an implant to be placed within the relatively small and isolated environment of the eye to avoid discomfort as the implant imbibes water. NHS had a significant effect on the rate of change in WAC under normal conditions (p=0.023) (FIG. 15a). The interaction between [NHS] and the catalyst [AlCl₃] had a notable effect on the WAC (p=0.060). The WAC was lowest at median levels of NHS and low levels of AlCl₃ (FIG. 17a).

As observed under normal conditions, the interaction between NHS and AlCl₃ also had a notable effect on the WAC observed when exposed to pathological conditions (p=0.066) (FIG. 15b). Here the imbibement of physiological fluids was limited when low [AlCl₃] and high [NHS] were employed (FIG. 17b). Higher AlCl₃ concentrations could potentially increase the hydrophilicity of the implant, and hence fluid uptake, due to potential incorporation of the electrolyte ions into the BPMs.

It is important that the device withstands stresses to which it is exposed, hence maintaining its resilience once implanted into the eye to avoid fragmentation and potential discomfort. The [NHS] significantly affected the resilience of the device when exposed to pathological conditions (p=0.047) (FIG. 15c). The resilience was most favourable at median levels of AlCl₃ (FIG. 17c).

### Response Optimization of the Device

Response optimization procedure (MINITAB®, V15, Minitab, USA) was used to obtain the optimised levels of the selected formulatory components. An optimal formulation was developed following simultaneous constrained optimization of MDT I N, MDT I F, Δ MDT I, Δ WAC N, and Δ WAC F. The optimized levels of the independent variables that would achieve the desired drug release and fluid uptake entrapment properties and their predicted responses were then determined. The optimised levels of the independent variables, the goal for the response, the predicted response, y, at the current factor settings, as well as the individual and composite desirability scores are shown in FIG. 18. Based on the statistical desirability function, it was found that the composite desirabilities for each of the formulations was 1.0. The constrained settings utilized are outlined in Table 7.

TABLE 7
Constrained settings for response optimization

| Parameter | Goal | Constraint |
|---|---|---|
| MDT I N | Minimise | 6-10 |
| MDT I F | Target | 12-18 |
| Δ MDT I | Maximise | 6-9 |
| Δ WAC N | Minimise | 15-20 |
| Δ WAC F | Maximise | −2.5-(−1.5) |

The ideal formulation was prepared according to the optimal predicted settings. The experimentally derived values for the responses of the optimal formulation were in close agreement with the predicted values (Table 8), demonstrating the reliability of the optimization procedure in predicting the bioresponsive behaviour of the device and ascertaining the significance of the effect of HA, NHS, DCC and AlCl₃ levels and their intricate interplay on the fluid uptake behaviour, with disentanglement of the crosslinked polymeric composite and ultimate drug release.

TABLE 8
Experimental and predicted response values
for the optimized formulations

| Measured | | | | | |
|---|---|---|---|---|---|
| Response | Predicted | Experimental | | R² | Desirability |
| MDT I N | 5.3584 | 5.6212 | | | 1.000 |
| MDT I F | 15.000 | 10.723 | | | 1.000 |
| Δ MDT I | 5.6416 | 5.1019 | {close oversize brace} | 0.9674 | 1.000 |
| Δ WAC N | 7.7296 | 7.8824 | | | 1.000 |
| Δ WAC F | −1.3925 | −1.2735 | | | 1.000 |

### Concurrent Optimization by ANN for Statistical Validation

In order to obtain accuracy and maximum degree of precision, the training was done twice (i.e. primary and secondary training). The leveling of the MSE with standard deviation (SD) boundaries for the training runs indicated a sequential improvement of data modeling as depicted in FIG. 19. Table 9 depicts the average of the MSE values for the three runs of the primary training, the best network run out 10,000 epochs, and the overall efficiency and performance of the neural network during the data training.

TABLE 9
Neural network indicators characterizing the efficiency
and performance of data in the primary training as per ANN

| Best Network | Training | Performance | Δ MDT I |
|---|---|---|---|
| Epoch # | 10000 | MSE | 3.972285765 |
| Minimum MSE | 0.005 | NMSE | 0.080975336 |
| Final MSE | 0.006 | MAE | 1.574042771 |
| | | Min Abs Error | 0.00837524 |
| | | Max Abs Error | 5.234174834 |
| | | R² | 0.958911855 |

MSE: Mean square error
NMSE: Normalized mean Square error
MAE: Mean absolute error
Min Abs Error: Minimum absolute error
Max Abs Error: Maximum absolute error

Based on the obtained results, it is evident that the employed training model was efficient (MSE=0.005). Results revealed a satisfactory fit for the input variables (R²=0.96). The performance criterion employed to assess the closeness and correlation between the desired and the actual network output for Δ MDT I of each formulation is depicted in FIG. 20. The sensitivity coefficient of each formulatory component (input variables) is depicted in FIG. 21. It is apparent that each variable considered had a fairly high sensitivity against the Δ MDT I. An optimum formulation based on each of the proposed formulatory components is thus desirable.

Kinetic Analysis of Drug Release from the Optimum Formulation

The kinetic models generated were in congruence with the bioresponsive capabilities of the device embodied by the polymeric transitions on exposure to normal and pathological fluids. The degree to which each model describes the optimum formulation is represented in Table 10. The release kinetics of both indomethacin and ciprofloxacin under normal conditions were best exemplified by the Higuchi model (R² of 0.9841 and 0.9892, respectively) indicating release of drug from the BPMs as a square root of time-dependent process based on Fickian diffusion. The Hixson Crowell cube root law was more applicable to the drug release kinetics of both indomethacin and ciprofloxacin under inflammatory conditions (R² of 0.9816 and 0.9906, respectively). This indicates the observed change in surface area and diameter of the implants with progressive bioresponsive erosion of the implants in the presence of hydroxyl radicals as a function of time. Furthermore, the release kinetics attained for indomethacin and ciprofloxacin under inflammatory conditions emulate a close fit with zero order release (R² of 0.9858 and 0.9903, respectively) in the presence of constant inflammation, which is the most desirable case for disease treatment. Korsemeyer and Peppas (Power law) was employed to provide a prediction of the drug release mechanism. Only release under inflammatory conditions fits the limits of this model, where n (representing the diffusion exponent) falls between 0.45 and 0.89, which is indicative of anomalous (non-Fickian) diffusion.

TABLE 19
Release parameters of an optimum device

| | Zero order | | Higuchi | | n | Peppas | | 1st order | | Hixson | |
|---|---|---|---|---|---|---|---|---|---|---|---|
| Form | k₀ (h⁻¹) | R² | k_{H} (h^{-1/2}) | R² | value | K_{KP} (h⁻ⁿ) | R² | k₁ (h⁻¹) | R² | k_{HC} (h^{-1/3}) | R² |
| Indo (N) | 0.0127 | 0.9408 | 0.4508 | 0.9841 | −0.4907 | 0.549 | 0.9892 | −6.00E−05 | 0.9451 | −0.0002 | 0.9437 |
| Indo (F) | 0.0407 | 0.9858 | 1.3737 | 0.9352 | −1.9224 | 1.1655 | 0.9697 | −0.0002 | 0.9791 | −0.0007 | 0.9816 |
| Cipro | 0.0103 | 0.9781 | 0.3594 | 0.9892 | 0.4695 | 0.2448 | 0.9855 | −5.00E−05 | 0.98 | −0.0002 | 0.9794 |
| (N) | | | | | | | | | | | |
| Cipro | 0.0306 | 0.9903 | 1.0535 | 0.9755 | 0.4611 | 0.3646 | 0.966 | −0.0002 | 0.9904 | −0.0006 | 0.9906 |
| (F) | | | | | | | | | | | |

### Lipo-Chitosan-Poly(ε-Caprolactone) Nanobubbles

The inflamed tissue-targeted nanobubbles displayed sizes ranging from 663 to 1869 nm (PdI=0.395). The zeta potential of the nanobubbles (+31.3 to +36.5 mV) attested to their enhanced stability and bioadhesive capabilities. Fourier-transform infrared spectroscopy studies confirmed the appropriate loading of indomethacin into the nanobubbles. Distinctive shifts in the molecular transitions were observed. The band representative of the carbonyl group of PCL was shifted to higher wavenumbers (from 1725 in native PCL to 1748 cm⁻¹) as well as a band at 1618 cm⁻¹ attributable to the hydrogen-bonded carbonyl groups with hydrogen-donating groups (—OH and —NH₂) of chitosan (FIG. 22). FIG. 23a depicts the chitosan-PCL nanogels. Incorporation of the nanogels into the medium molecular weight-based chitosan matrix elaborated progressive coating of the nanogels (FIGS. 23b and c). The inflamed tissue-targeted systems ultimately boasted a ‘star-like’ appearance, which, upon release from the crosslinked core matrix, could facilitate mucoadhesion of the positively charged particles onto the negatively charged membranes within the eye. FIG. 24 depicts the lipo-chitosan-PCL nanobubbles maintained within the inner BPM composed of chitosan. Hydrolysable linkages are established between the matrix and nanobubbles which ultimately release the nanobubbles on exposure to dissolution media. The hydrolysis is anticipated to occur to a greater extent on exposure to inflammatory mediators (i.e. hydroxyl radicals) owing to the described responsive behaviour of chitosan. Further ex vivo studies through excised New Zealand Albino rabbit ocular sections, not provided herein, have highlighted the potential of the designed nanosystem to adequately penetrate ocular barriers such as the BRB, for efficient delivery of the therapeutic load to the posterior segment ocular tissues.

### Outer Bioresponsive Polymeric Matrix

The underlying molecular mechanisms emanating in the formation of the interpenetrating crosslinked BPMs with drug involvement is depicted in FIG. 25 and the observed chemical transitions for the outer BPM in FIG. 26. There is the ultimate presence of two distinct BPMs displaying the highlighted differential drug release characteristics.

### CONCLUSION

[NHS] and [AlCl₃] had a significant or notable effect on the MDT of indomethacin under normal and pathological conditions, respectively (p=0.048; p=0.058). The interaction between the inflammation-responsive [HA] and [DCC] emanated in a significant effect on the ΔMDT of indomethacin (p=0.050). [AlCl₃] also had a significant impact on the WAC of the device under normal conditions (p=0.023), whereas the effect of [NHS] was significant when considering the resilience of the device under pathological conditions (p=0.047). Subsequent execution of ANN with further training of the data confirmed the adequacy of the design. Analysis of the drug release kinetics from the optimum device under both normal and pathological conditions was in coherence with the anticipated behaviour of an inherently bioresponsive device.

The drug release pattern obtained from the device thus differs considerably from that reported for the market leader, Retisert™. Furthermore, surgical complications (e.g. choroidal detachment, endophthalmitis, hypotony, retinal detachment, vitreous hemorrhage, vitreous loss, exacerbation of intraocular inflammation and wound dehiscence) (Ahn and Moshfeghi, 2008) would be minimized in the device due to the biodegradability of the device, avoiding the need for removal of the device which is necessitated in non-biodegradable implants such as Retisert™.

### REFERENCES

1. Resnikoff S, Pascolini D, Etya'ale D, Kocur I, Pararajasegaram R, at al. Global data on visual impairment in the year 2002. Bull World Health Organ 2004, 82: 844-851.
2. Mathenge W, Kuper H, Polack S, Onyango O, Nyaga G, et al. Rapid assessment of avoidable blindness in Nakuru district, Kenya. Ophthalmol 2007, 114: 599-605.
3. Wadud Z, Kuper H, Polack S, Lindfield R, Rashid Akm M, et al. Rapid assessment of avoidable blindness and needs assessment of cataract surgical services in Satkhira district, Bangladesh. Br J Ophthalmol 2006, 90: 1225-1229.
4. Oye J E, Kuper H, Dineen B, Befidi-Mengue R. Foster A ( ) Prevalence and causes of blindness and visual impairment in Muyuka: A rural health district in South West Province, Cameroon. Br J Ophthalmol 2006, 90: 538-542.
5. Ngondi J, Ole-Sempele F, Onsarigo A, Matende I, Baba S, et al. Prevalence and causes of blindness and low vision in Southern Sudan. PloS Medicine. 3 doi:10.1371/journal.pmed.0030478, 2006.
6. Nanjawade B K, Manvi F V, Manjappa A S. In situ-forming hydrogels for sustained ophthalmic drug delivery. J Contr Rel 2007, 122(2): 119-134.
7. Herrero-Vanrell R, Refojo M F. Biodegradable microspheres for vitreoretinal drug delivery. Adv Drug Del Rev 2001, 52(1): 5-16.
8. Del Amo E M, Urtti A Current and future ophthalmic drug delivery systems: A shift to the posterior segment. Drug Discovery Today 2008, 13(3-4): 135-143.
9. Yasukawa T, Ogura Y, Sakurai E, Tabata Y, Kimura H. Intraocular sustained drug delivery using implantable polymeric devices. Adv Drug Del Rev 2005, 57(14): 2033-2046.
10. Rao N A. Role of oxygen radicals in retinal damage associated with experimental uveitis Tr Am Opth Soc 1990, 87:797-850.
11. Champagne E S. Ocular Pharmacology. Clin Tech Small Anim Prac 2001, 16(1): 13-16.
12. Holekamp N M, Thomas M A, Pearson A. The safety profile of long-term, high-dose intraocular corticosteroid delivery. Am J Ophthalmol 2005, 139(3): 421-428.
13. Reichle M L. Complications of intravitreal steroid injections. Optometry 2005, 76(8): 450-460.
14. Jonas J B, Sofker A. Intraocular injection of crystalline cortisone as adjunctive treatment of diabetic macular edema. Am J Ophthalmol 2001, 132: 425-427.
15. Jonas J B, Kreissig I, Degenring R F. Intravitreal triamcinolone acetonide for pseudophakic cystoid macular edema. Am J Ophthalmol 2003, 136: 384-386.
16. Park C H, Jaffe G J, Fekrat S. Intravitreal triamcinolone acetonide in eyes with cystoid macular edema associated with central retinal vein occlusion. Am J Ophthalmol 2003, 136: 419-425.
17. Young S, Larkin G. Branley M, Lightman S. Safety and efficacy of intravitreal triamcinolone for cystoid macular oedema in uveitis. Clin Exp Ophthalmol 2001, 29: 2-6.
18. Jonas J B, Hayler J K. Panda-Jonas S. Intravitreal injection of crystalline cortisone as adjunctive treatment of proliferative vitreoretinopathy. Br J Ophthalmol 2000, 84: 1064-1067.
19. Callegan M C, Gilmore M S, Gregory M, Ramadan R T, Wiskur B J, Moyer A L, Hunt J J, Novosad B D. Bacterial endophthalmitis: Therapeutic challenges and host-pathogen interactions. Prog Ret Eye Res 2007, 28(2): 189-203.
Roth D B, Chieh J, Spim, Green S N, Yarian D L, Chaudhry N A. Noninfectious endophthalmitis associated with intravitreal triamcinolone injection. Arch Ophthalmol 2003, 121: 1279-1282.
21. Moshfeghi D M, Kaiser P K, Scott I U et al. Acute endophthalmitis following intravitreal triamcinolone acetonide injection. Am J Ophthalmol 2003, 136: 791-796.
22. Ahn B J, Moshfeghi A A Implantable Posterior Segment Drug Delivery Devices. Ophthalmology Web 2008. http://www.ophthalmologyweb.com/Spotlight.aspx?spid=23&aid=253&headerid=23.
23. Jonas J B, Hayler J, Sofker A, Panda-Jonas S. Intravitreal injection of crystalline cortisone as adjunctive treatment of proliferative diabetic retinopathy. Am J Ophthalmol 2001, 131: 468-471.
24. Ligório Fialho S, Behar-Cohen F, Silva-Cunha A. Dexamethasone-loaded poly(ε-caprolactone) intravitreal implants: A pilot study. Eur J Pharm Biopharm 2008, 68(3): 637-46.
25. Alvarez-Lorenzo C, Concheiro A. Molecularly imprinted polymers for drug delivery. J Chrom B 2004, 804(1): 231-245.
26. Barbu E, Verestiuc L, Novell T G, Tsibouklis J. Polymeric materials for ophthalmic drug delivery: trends and perspectives. J Mater Chem 2006, 16: 3439-3443.
27. Panyam J. Inflammation-responsive drug delivery system. Pharmaceutical Sciences, WSU_.htm. http://research.wayne.edu/dre/db/?view=person&id=42. Last retrieved 2 Feb. 2008.
28. Bourges J-L, Gautier S E, Delie F et al. Ocular Drug Delivery Targeting the Retina and Retinal Pigment Epithelium Using Polylactide Nanoparticles. Invest Ophthalmol Vis Sci 2003, 44: 3562-3569.
29. C L Hawkins, Davies M J. Direct detection and identification of radicals generated during the hydroxyl radical-induced degradation of hyaluronic acid and related materials. Free Radical Biol Med 1996, 21: 275-290
30. Holekamp N M, Thomas M A, Pearson A. The safety profile of long-term, high-dose intraocular corticosteroid delivery. Am J Ophthalmol 2005, 139(3): 421-428.
31. Yui N, Nihira J, Okano T, Sakurai Y. Inflammation responsive degradation of crosslinked hyaluronic acid gels. J Contr Rel 1992, 22: 105-116.
32. Sahoo S K, Dilnawaz F, Krishnakumar S. Nanotechnology in ocular drug delivery. Drug Discovery Today 2008, 13(3-4): 144-151.
33. Li W, Huang Z, MacKay J A, Grube S, Szoka Jr F C. Low-pH-sensitive poly(ethylene glycol) (PEG)-stabilized plasmid nanolipoparticles: effects of PEG chain length, lipid composition and assembly conditions on gene delivery. J Gene Med 2005, 7: 67-79.
34. Sastry S V, Reddy I K, Khan M A: Atenolol gastrointestinal therapeutic system: optimization of formulation variables using response surface methodology. J Contr Rel 1997, 45(2): 121-130.
35. Scott J E, Tigwell M J. Periodate-induced viscosity decreases in aqueous solutions of acetal- and ether-linked polymers. Carbohyd. Res. 28 (1973), p. 53.

1. An implantable device for the in situ delivery of one or more pharmaceutically active agents to a human or animal, the device comprising:
an outer polymeric matrix formed from at least one inflammation-sensitive polymer and comprising at least one pharmaceutically active agent, wherein the polymer is cross-linked so as to retain the pharmaceutically active agent within the polymeric matrix under normal physiological conditions but undergoes a conformational change when inflammation is present so as to release the pharmaceutically active agent; and
an inner polymeric matrix formed from at least one inflammation-sensitive polymer and comprising at least one pharmaceutically active agent, wherein the polymer is cross-linked so as to retain the pharmaceutically active agent within the polymeric matrix under normal physiological conditions but undergoes a conformational change when inflammation is present so as to release the pharmaceutically active agent;
wherein the inner and outer polymeric matrices are formed so that, when inflammation is present the pharmaceutically active agent within the inner polymeric matrix is released at a slower rate than the pharmaceutically active agent within the outer polymeric matrix.

2. The device according to claim 1, which is an intraocular device for implantation into the eye.

3. The device according to claim 1, wherein the outer polymeric matrix provides fast to intermediate release of the pharmaceutically active agent for the therapeutic management of infection and/or preliminary inflammation, and the inner polymeric matrix provides slower release of the pharmaceutically active agent for chronic responsive management of inflammation.

4. The device according to claim 1, wherein the inner polymeric matrix is chemically modified by cross-linking to provide a slower release rate of the pharmaceutically active agent than the cuter polymeric matrix.

5. The device according to claim 1, which is biodegradable.

6. The device according to claim 1, wherein the polymer of the inner polymeric matrix is a cationic low-molecular weight carbohydrate polymer.

7. The device according to claim 1, wherein the polymer of the inner polymeric matrix is chitosan.

8. The device according to claim 1, wherein the polymer of the outer polymeric matrix is an anionic polymer or a mixture of anionic polymers.

9. The device according to claim 1, wherein the polymer of the outer polymeric matrix is hyaluronic acid.

10. The device according to claim 1, wherein the polymers of the inner and outer polymeric matrices are eroded by free radicals released from activated leukocytes during acute and chronic intraocular inflammatory reactions.

11. The device according to claim 1, wherein each of the polymeric matrices is cross-linked with gluteraldehyde.

12. The device according to claim 11, wherein each of the polymeric matrices is double-crosslinked employing carbodiimide coupling chemistry.

13. The device according to claim 9, wherein the outer polymeric matrix further comprises alginate, polygalacturonate, methylcellulose (polyacetals), poly (ethylene) oxide or poly (acrylic acid).

14. The device according to claim 13, wherein the ratio of alginate:hyaluronic acid is about 16:1 and the ratio of alginate:poly (acrylic acid) is about 4:1.

15. The device according to claim 11, wherein the ratio of chitosan to the gluteraldehyde cross-linking agent in the inner polymeric matrix is about 7:1.

16. The device according to claim 1, wherein the pharmaceutically active agent of the outer polymeric matrix is an antibiotic.

17. The device according to claim 1, wherein the pharmaceutically active agent of the outer polymeric matrix is an anti-inflammatory agent.

18. The device according to claim 1, wherein the pharmaceutically active agent of the inner polymeric matrix is an anti-inflammatory agent.

19. The device according to claim 1, wherein the pharmaceutically active agent in the inner polymeric matrix is within or on nanoparticles.

20. The device according to claim 19, wherein the nanoparticles are formed from poly(ε-caprolactone), chitosan, phospholipids and the pharmaceutically active agent.

21. The device according to either of claim 19 or claim 20, wherein the nanoparticles are nanobubbles.

22. The device according to claim 1, wherein the anti-inflammatory agent is indomethacin.

23. The device according to claim 16, wherein the antibiotic is ciprofloxacin.

24. The device according to claim 1, which defines at least one aperture for suturing the implant to a site in the body.

25. The device according to claim 1, which is for use in preventing or treating inflammatory or infectious conditions in the eye.

26. A device according to claim 1, which is for use in preventing or treating inflammatory or infectious conditions selected from the group consisting of HIV/AIDS, influenza, arthritis, lupus, fibromyalgia, juvenile rheumatoid arthritis, osteomyelitis and septic (infectious) arthritis.

27. A device according to claim 1, which comprises:
an outer polymeric matrix formed from cross-linked hyaluronic acid and comprising an antibiotic, wherein the outer polymeric matrix is eroded when inflammation is present and releases the antibiotic; and
an inner polymeric matrix formed from cross-linked chitosan and comprising nanoparticles which comprise an anti-inflammatory agent, wherein the inner polymeric matrix is eroded when inflammation is present and releases the nanoparticles with the anti-inflammatory agent;
wherein the outer and inner polymeric matrices are prepared so that the pharmaceutically active agent within the inner polymeric matrix, is released at a slower rate than the pharmaceutically active agent within the outer polymeric matrix.

28. A method of manufacturing a device according to claim 1, the method comprising the steps of:
forming nanoparticles from poly(ε-caprolactone), chitosan, phospholipids and a pharmaceutically active agent;
forming an inner polymeric matrix from the nanoparticles and a polymer which erodes when exposed to inflammation;
forming an outer polymeric matrix from a pharmaceutically active agent and a polymer which erodes when exposed to inflammation, wherein the outer polymeric matrix is designed to erode at a faster rate than the inner polymeric matrix when exposed to inflammation, and so to release the pharmaceutically active agent from the outer polymeric matrix at a faster rate than the inner polymeric matrix;
placing the inner polymeric matrix in an inner portion of a mould;
placing the outer polymeric matrix in an outer portion of the mould;
drying the matrices to form a solid device which is suitable for implantation; and
optionally creating apertures in the device to enable it to be sutured to a site in the body.

29. A method of treating infection and/or inflammation in a human or animal, the method comprising inserting a device as claimed in claim 1 into the human or animal, wherein:
an outer polymeric matrix of the device releases, in the presence of inflammation, a therapeutically effective amount of an antibiotic to treat the infection and preliminary inflammation; and
an inner polymeric matrix of the device releases a therapeutically effective amount of an anti-inflammatory agent at a slower rate than the outer polymeric matrix to treat a chronic inflammatory condition.

30. The method according to claim 29, wherein the infection and/or inflammation is in an eye and the device is inserted into the posterior segment (at the pars plana), or sub-Tenon, or intrasclerally, or on the sclera of the eye.
